(19) United States
(12) Patent Application Publication
(10) Pub. No.: US 2008/0213189 A1
(43) Pub. Date: Sep. 4, 2008

(54) Multifunctional metal-graphite nanocrystals

(75) Inventors: Jin Hyung Lee, Mountain View, CA (US); Won-Seok Seo, Seoul (KR); Hongjie Dai, Cupertino, CA (US); Zhuang Liu, Stanford, CA (US); Sarah Paige Sherlock, New Haven, VT (US)

Correspondence Address: PETERS VERNY , L.L.P., 425 SHERMAN AVENUE, SUITE 230, PALO ALTO, CA, 94306, US

(73) Assignee: The Board of Trustees of the Leland Stanford Junior University

(21) Appl. No.: 11/975,008

(22) Filed: Oct. 16, 2007

Related U.S. Application Data
(60) Provisional application No. 60/852,531, filed on Oct. 17, 2006.

(51) Int. Cl. A61K 49/06; B32B 5/16; C12N 5/06; A61P 35/00; A61K 33/00; C12N 13/00

(52) U.S. Cl. 424/9.32; 428/403; 435/325; 435/173.1; 424/600

(57) ABSTRACT

Disclosed are nanocrystals comprising metals and metal alloys, which are formed by a process that results in a layer of graphite in direct contact with the metallic core. The nanocrystals may be used in vivo as MRI contrast agents, X-ray contrast agents, near IR (NIR) heating agents, drug delivery, protein separation, catalysis etc. The nanocrystals may be further functionalized with a hydrophilic coating, e.g., phospholipid-polyethylene glycol, which improves in vivo stability. The process comprises chemical vapor deposition of metals adsorbed onto silica as a fine powder, in conjunction with a carbon containing gas, which coats the metal particles. The silica is then etched away. Preferred metals include iron, gold, cobalt, platinum, ruthenium and mixtures thereof, e.g., FeCo and AuFe. The process permits control of the alloy compositions, size, and other characteristics.

### CROSS-REFERENCE TO RELATED APPLICATIONS

This application claims priority from U.S. Provisional Patent Application No. 60/852,531, filed on Oct. 17, 2006, which is hereby incorporated by reference in its entirety.

### STATEMENT OF GOVERNMENTAL SUPPORT

This invention was made with U.S. Government support under NIH Grant no. 1 U54 CA119367-01. The U.S. Government has certain rights in this invention.

### REFERENCE TO SEQUENCE LISTING, COMPUTER PROGRAM, OR COMPACT DISK

None.

### BACKGROUND OF THE INVENTION

1. Field of the Invention

The present invention relates to the field of metallic nanocrystals, having graphitic shells, their chemical modification and their use in vivo in such fields as drug delivery, cellular labeling, contrast agents for imaging, etc.

2. Related Art

Nanocrystals with advanced magnetic or optical properties have been actively pursued for potential biological applications including imaging, diagnosis, therapy and their integration.4-12

Nanocrystals can be prepared from a wide variety of metals and metal alloys.

Among various magnetic nanocrystals,^{2,13-17} FeCo has yet to be widely explored despite superior magnetic properties.³ Described below is the synthesis of FeCo/single graphitic-shell nanocrystals using a scalable method. Multi-functionality of FeCo nanocrystals is demonstrated by utilizing magnetic properties of the FeCo core and high NIR optical absorbance of the single-layered graphitic shell. Previously, FeCo nanocrystals with multi-layered graphitic carbon,¹⁸ pyrolytic carbon,¹⁷ or inert metals¹⁹ have been obtained, but not in single-shelled, discrete, chemically functionalized and water-soluble forms desired for biological applications.

FeCo alloy is unique among magnetic materials with superior properties.¹ Thus far, degradation of nanocrystalline FeCo due to oxidation and potential toxicity of Co have prevented utilization of FeCo in biological settings.2, 3 For example, Turget et al., “Magnetic properties and microstructural observations of oxide coated FeCo nanocrystals before and after compaction,” J. App. Phys. 85:4406-4408 (1999) describes such nanocrystals, which have an oxide coating.

Nanocrystals according to the present method are preferably prepared by chemical vapor deposition (CVD).

(CVD) refers to chemical reactions that transform gaseous molecules, called precursor, into a solid material, in the form of thin film or powder, on the surface of a substrate. Described below is the synthesis of core-shell FeCo nanocrystals with a single-layer of graphitic-carbon shell, a material believed to be novel. These nanocrystals exhibit the highest saturation magnetization, high optical absorbance in the near-infrared (NIR) by the graphitic shell, and remarkable chemical stability. The FeCo/graphitic carbon (FeCo/GC) nanocrystals are functionalized by phospholipid-polyethylene glycol for water solubility and enabling biological applications of FeCo. Multi-functionality is demonstrated including non-toxic labeling of stem cells as contrast agent for magnetic resonance imaging (MRI) with unprecedented high relaxivities and NIR agents for opto-thermal manipulation. Further, the FeCo/GC nanocrystals afford positive contrast enhancement for T₁ weighted MRI owing to unusually high r1 relaxivities. Thus, nanocrystals with integrated magnetic and optical properties shall enable new possibilities in biological imaging, manipulation and therapy.

The present materials have been studied for use as MRI contrast agents.

In MRI, the image of an organ or tissue is obtained by placing a subject in a strong external magnetic field and observing the effect of this field on the magnetic properties of the protons (hydrogen nuclei) contained in and surrounding the organ or tissue. The proton relaxation times, termed T₁ and T₂, are of primary importance. T₁ (also called the spin-lattice or longitudinal relaxation time) and T₂ (also called the spin-spin or transverse relaxation time) depend on the chemical and physical environment of organ or tissue protons and are measured using the RF (radio frequency) pulsing technique; this generates signal that is dependent on many parameters that include T₁ and T₂. The pulsing is controlled in a way that the resulting signals can be analyzed to generate spatially resolved images with contrast dependent on T₁ and T₂. By generating images with various pulsing methods, spin-lattice relaxation rate R₁ (1/T₁) and the spin-spin relaxation rate R₂ (1/T₂) can be calculated.

The image produced, however, often lacks definition and clarity due to the similarity of the signal from different tissues. To generate an image with good definition, T₁ and/or T₂ of the tissue to be imaged must be distinct from that of the background tissue. In some cases, the magnitude of these differences is small, limiting diagnostic effectiveness. Thus, there exists a real need for methods that increase or magnify these differences. One approach is the use of contrast agents.

As any material suitable for use as a contrast agent must affect the magnetic properties of the surrounding tissue, MRI contrast agents can be categorized by their magnetic properties. Macromolecular paramagnetic contrast agents are being tested worldwide. Preclinical data shows that these agents demonstrate great promise for improving the quality of MR angiography, and in quantifying capillary permeability and myocardial perfusion.

Ultrasmall superparamagnetic iron oxide (USPIO) particles have been evaluated in multicenter clinical trials for lymph node MR imaging and MR angiography, with the clinical impact under discussion. In addition, a wide variety of vector and carrier molecules, including antibodies, peptides, proteins, polysaccharides, liposomes, and cells have been developed to deliver magnetic labels to specific sites.

For MRI, two different classes of contrast agents exist: agents that influence mainly the signal in T₂—(negative contrast agents, reducing the signal) or in T₁-weighted images (positive contrast agents, increasing the signal). Typical T₁ contrast agents are small molecular weight compounds containing a single Lanthanide chelate as contrast-producing element (e.g., Gadolinium-DTPA). The tissue concentration necessary to image with these T₁ contrast agents on a molecular level is considerably higher than the required concentration of iron oxide particles; it has to be in the order of mMols, since T₁ relaxivity values (r₁) are usually only in the 5-10 (mMs)-1 range. Gd (III), with 7 unpaired electrons and a long electronic relaxation time, is commonly used in such agents. However, current Gd (III)-based commercial agents have relatively poor contrast enhancement capabilities or low sensitivity due to their low relaxivity. Another important limitation as an intravascular Gd molecular agent currently used in the clinic is that its blood circulation time is very short. It starts leaking out into the tissue in seconds, reducing the blood, tissue contrast (which is important for imaging blood vessels).

For T₂ contrast, the most common materials are iron oxide nanoparticles—Superparamagnetic Iron Oxide (SPIO), Very Small Paramagnetic Iron Oxide (VSPIO) or Ultrasmall Superparamagnetic Iron Oxide (USPIO). These particles usually consist of a crystalline iron oxide core, surrounded by polymer coating, often dextran, polyethyleneglycol, or ionic sheath (e.g., citrate). The advantage of these preparations is that each particle contains thousands of iron atoms, resulting in very high T₂ relaxivities of up to 200 (mMs)-1, which makes detection of lower concentrations of contrast agents (μmol to nmol range) possible.

### Patents and Publications

Wang et al., “Magnetic resonance tracking of nanoparticle labeled neuronal stem cells in a rat's spinal cord,” Nanotechnology 17:1911-1915 (15 Mar. 2006) discloses the use of superparamagnetic gold-coated monocrystalline iron oxide nanoparticles intended for use as contrast enhancers.

US 2002/0178846 by Dai, et al., published Dec. 5, 2002, entitled “Carbon nanotubes and methods of fabrication thereof using a catalyst precursor,” discloses dip coating of silicon pyramids with a liquid phase catalyst followed by chemical vapor deposition (CVD) using methane for growing SWNTs.

Kulkami et al., “Mesoscale organization of metal nanocrystals,” discloses two-dimensional arrays formed by nanocrystals of metals covered by alkanethiols. Nanoparticles of various sizes of Pd, Ag, Cd and Au have been reported. The authors examined the structure and stability of Pd nanocrystals (e.g., 4.5 nm) obtained using alkanethiols (e.g., butanethiol, etc., which resulted in a lattice formation) and PVP as surfactants. The authors also studied Pd561Nix core shell nanocrystals.

Wang et al., “A general strategy for nanocrystal synthesis,” Nature 437, 121-124 (1 Sep. 2005) discloses the synthesis of nanocrystals with different chemistries and properties and with low dispersity; these include noble metal, magnetic/dielectric, semiconducting, rare-earth fluorescent, biomedical, organic optoelectronic semiconducting and conducting polymer nanoparticles. The reactions were controlled at different temperatures for specific metals, for example, 80 to 200° C. for Ag, 20 to 200° C. for Ru, Rh and Ir, 20 to 100° C. for Au, Pd and Pt. The primary reaction in the preparation of noble metal nanocrystals through this liquid-solid-solution (LSS) process involved the reduction of noble metal ions by ethanol at the interfaces of metal linoleate (solid), ethanol-linoleic acid liquid phase (liquid) and water-ethanol solutions (solution) at different designated temperatures.

Babincova, et al., “In vivo heating of magnetic nanoparticles in alternating magnetic field,” Med. Phys. 31 (2004) 2219 discloses magnetic nanoparticles.

Ingrid Hilger et al, “Magnetic nanoparticles for selective heating of magnetically labeled cells in culture: preliminary investigation,” 2004 Nanotechnology 15 1027-1032, reports work where using mouse endothelial cells in culture, the binding of dextran coated magnetic nanoparticles (mean hydrodynamic particle diameter 65 nm) was modeled using the periodate method. The amount of iron immobilized on cells was found to be 153±56 μg Fe per 1×107 cells as determined by atomic absorption spectrometry.

U.S. Pat. No. 6,133,047 to Elaissari, et al., issued Oct. 17, 2000, entitled “Superparamagnetic monodisperse particles,” discloses superparamagnetic monodispersed particles comprising a core of a first polymer, an internal layer of a second polymer coating the core and in which a magnetic material (iron oxide) is distributed, and an external layer of a third polymer coating the magnetic layer and capable of interacting with at least one biological molecule.

Y P He et al., “Synthesis and characterization of functionalized silica-coated Fe₃O₄ superparamagnetic nanocrystals for biological applications,” 22 Apr. 2005 J. Phys. D: Appl. Phys. 38 1342-1350 disclose superparamagnetic Fe₃O₄ nanocrystals prepared by a chemical coprecipitation method with a thin thickness-adjustable silica layer coated on the surface by hydrolysis of tetraethyl orthosilicate. The silica-coated Fe₃O₄ nanocrystals consisted of a 6-7 nm diameter magnetic core and a silica shell about 2 nm thick, according to transmission electron microscopy observations.

Bao et al., “Controlled self-assembly of colloidal cobalt nanocrystals,” Journal of Magnetism and Magnetic Materials 266 (2003) L245-L249 discloses using surfactant-stabilized Co nanoparticles exhibiting superparamagnetic behavior.

U.S. Pat. No. 4,951,675, “Biodegradable superparamagnetic metal oxides as contrast agents for MR imaging,” issued Aug. 28, 1990, discloses a dextran-coated iron oxide particle dispersoid which is injected into a subject's bloodstream, and the particles localize in the liver.

Huh et al., “In vivo magnetic resonance detection of cancer by using multifunctional magnetic nanocrystals,” J Am Chem Soc. 2005 Sep. 7; 127(35):12387-91 discloses magnetic nanocrystals conjugated to a cancer-targeting antibody, Herceptin, and subsequent utilization of these conjugates as MRI probes.

Flacke et al., “Novel MRI Contrast Agent for Molecular Imaging of Fibrin,” Circulation. 104:1280-1285 (2001) discloses nanoparticles were formulated with 2.5 to 50 mol % Gd-DTPA-BOA, which corresponds to >50 000 Gd³⁺ atoms/particle. Paramagnetic nanoparticles were characterized in vitro and evaluated in vivo. In contradistinction to traditional blood-pool agents, T₁ relaxation rate as a function of paramagnetic nanoparticle number was increased monotonically with Gd-DTPA concentration from 0.18 mL·s⁻¹·pmol⁻¹ (10% Gd-DTPA nanoparticles) to 0.54 mL·s⁻¹·pmol⁻¹ for the 40 mol % Gd-DTPA formulations.

### BRIEF SUMMARY OF THE INVENTION

The following brief summary is not intended to include all features and aspects of the present invention, nor does it imply that the invention must include all features and aspects discussed in this summary.

The present invention is directed to a nanoparticle in the form of a nanocrystal comprising at least one metal in at least one layer (preferably 1-2 atomic layers) of a graphitic shell. The nanocrystal is generally less than about 20 nm nominal diameter, preferably equal to or less than 7 nm nominal diameter and equal to or greater than 4 nm, depending on the metal(s) used. The graphitic shell directly contacts the metal, and the metal is substantially free of oxygen or any other chemical attack due to the tight protection by the ultra-inert graphite coating.

By “substantially free,” it is meant that there are no oxygen or oxide atoms in contact with the metal, although a trace amount of such impurity may be tolerated, so long as the integrity of the complete graphitic coating is not disturbed and the magnetic property is not degraded. Degradation of FeCo magnetic properties by oxygen is discussed in Chaubey et al., “Synthesis and Stabilization of FeCo Nanoparticles,” J. Am. Chem. Soc. 2007, 129, 7214-7215.

The metal for the nanocrystal may be varied, but is preferably selected from the group consisting of Fe, Co, Ni, Au, Pt, FeCo, FeNi, FeAu, FePt, FeRu, CoRu, FeCoRu, FeCoPt, and CoPt. The metal nanocrystal may be 1 to 3 different metallic elements. In one aspect, the nanocrystal metal is of the formula X, Y and optionally Z, where X, Y and Z are independently selected from the group consisting of: Fe, Co, Ni, Au, Pt, Co, Ni, Ru, and Pt. X, Y and Z may be the same or different, meaning that the metal nanocrystal may be a single metal, or an alloy of two or three metals. In one aspect, metal nanocrystal may be selected from one or more of a Group VIII transition metal (Fe, Co, Ni, Ru, Rh, Pd, Os, Ir, Pt, Au), or from the group consisting of Fe, Co, Ni, Au, Pt, FeCo, FeNi, FeAu, and FePt. In one preferred embodiment, the nanocrystal is either FeAu. FeRu, or FeCo. The Fe to other metal (e.g., Co) ratio may be between 0:100 and 100:0.

The metal component will have superior magnetic properties for an MRI contrast agent. The graphitic shell generally comprises between 1 and 10, preferably between 1 and 2, graphitic layers comprised of sp² carbon atoms, where sp² bonding is characteristic of graphitic carbon.

The nanocrystal is preferably superparamagnetic to give optimum MRI contrast properties, but may also be ferromagnetic, based on the size and metal composition.

The nanocrystal may be functionalized for nonaggregation in an aqueous environment in that it is provided with a coating of organic molecules. The coating need not be completely covering the shell. However, the coating will help keep the nanocrystals dispersed and allows them to be formulated in a stable suspension. The organic molecules may be organic amphiphilic molecules noncovalently bound to the graphitic shell. For example, a lipid portion is adsorbed onto the shell surface, and a hydrophilic portion extends into an aqueous medium for solubility. A polar lipid contacting the graphitic shell may be used for this purpose, and the polar lipid may be linked to PEG. The adsorption is strong due to hydrophobic interaction between the graphitic layer and the aliphatic lipid. The PEG may have a molecular weight between 200 and 20,000, or even 50,000 Daltons. Experimental data have been obtained and are presented below with 5 kD and 2 kD PEG chains, and the longer chains are thought to improve particle solutions. Other hydrophilic polymers, which may be used are PEG, dextran or polyglycerol. It has been found experimentally that 3 arm, 4 arm and 6 arm branched PEGs all work with the present metal-graphite nanocrystals. The molecular weights of these PEGs ranged from 1 kD to 50 kD. The synthesis of branched PEG is described, for example, in U.S. Pat. No. 6,113,906 to Greenwald, et al., issued Sep. 5, 2000, entitled “Water-soluble non-antigenic polymer linkable to biologically active material.” Branched PEG prepared in various activated forms is supplied by Nektar Advanced PEGylation, Pierce Biotechnology, NOF Corporation and others.

The particles are prepared in a suspension without aggregation. Instead of having a coating molecule adsorbed on the nanocrystal, it may be covalently bonded to the graphitic shell. Particularly preferred, and exemplified below are nanocrystals made from AuFe and FeCo, as binary alloys. The metal in the present nanocrystals may have a very well defined local atomic arrangement but lack the extended order of usual crystals, or they may be entirely a single crystal. They may be chosen to have superior magnetic saturation for use, e.g., as MRI contrast agents, that is, at least 160 emu/g and up to the bulk value of FeCo alloy.

The present nanocrystals, when coated with an organic molecule, may be used in vitro to label cells, or they may be used to heat cells when near radiation is applied. Near infrared radiation transmits well through biological tissue, but is absorbed by the graphitic carbon shell, thereby heating the nanocrystal and the surrounding material. Also, as is known, an alternating magnetic filed may be applied to magnetic nanoparticles to heat the nanoparticles and the surrounding medium.

The present nanocrystals have also been used to deliver drugs and have shown tumor reduction in animal models. For this, one uses a dispersed nanocrystal comprising metal in at least one graphitic shell for delivery of an active agent into a cell. Dispersion is achieved by functionalization. The metal graphite nanocrystal comprises a hydrophilic polymer bound to the graphitic shell; and an active agent, attached to the nanocrystal through the graphitic shell. This functionalization may be achieved by a hydrophilic polymer, which is in turn bound to the active agent. The active agents exemplified are small molecule anti-cancer agents, but other active agents may be used, as described below. The active agent may be linked through the hydrophilic polymer by a number of known coupling chemistries.

Also demonstrated is a method of imaging blood flow in a blood vessel by applying a magnetic field to a dispersed preparation of metal-graphite nanocrystals, which have been linked to a hydrophilic polymer and injected into the blood vessel. The present agents have been shown to achieve very high resolution in MRI studies, allowing visualization of individual blood vessels well below 1 mm in diameter. Also provided is a method of reducing tumor size in a subject having a tumor, comprising the step of administering to the subject a solubilized metal-graphite nanocrystal linked to an anti-tumor drug.

### BRIEF DESCRIPTION OF THE DRAWINGS

FIG. 1 is a schematic drawing of several embodiments of nanocrystals according to the present invention and their method of synthesis;

FIG. 2 shows in FIG. 2A and FIG. 2B structural analysis of FeCo/GC (graphitic carbon) nanocrystals, as represented by TEM images of ˜7 nm and ˜4 nm nanocrystals respectively; FIG. 2C shows a selected area electron diffraction pattern of ˜7 nm nanocrystals. Note that the largest nanocrystals synthesized were ˜10 nm in mean diameter by increasing the metal precursor loading on silica support. The average sizes of ˜4 nm, ˜7 nm and ˜10 nm were all below the superparamagnetic limit of ˜20 nm (this high limit is due to the low magnetic anisotropy of FeCo, see ref. 1-3); FIG. 2D is a graph showing powder XRD (x-ray diffraction) data for ˜7 nm and ˜4 nm nanocrystals respectively; the small peaks marked by * on the XRD curve of ˜4 nm nanocrystals are assigned to fcc-CoFIg. FIG. 2E is a micrograph representing a high resolution TEM image of two FeCo nanocrystals each with a graphitic shell (pointed by arrow); FIG. 2F is a Raman spectrum (excitation 785 nm) of ˜7 nm FeCo/GC nanocrystals showing the G and D band of graphitic carbon.

FIG. 3A is a graph showing room temperature magnetization vs. field data for ˜7 nm (boxed line) and ˜4 nm (no box line) nanocrystals measured shortly after synthesis, and after one month exposure in ambient air for the ˜7 nm nanocrystals (boxes show no degradation from the solid black line). Note that no hysteresis loop exists in the solid lines comprised of data of cycling the field, due to superparamagnetism of the nanocrystals; FIG. 3B is a schematic of a FeCo/GC nanocrystal, structure of the phospholipid molecule used for functionalization, and a photograph showing a clear PBS suspension of functionalized FeCo nanocrystals taken after heating to 80° C. for 1 h. FIG. 3C is a graph of UV-vis-NIR absorption spectra of PBS suspensions of ˜7 nm functionalized nanocrystals at various molar metal concentrations (values in parentheses are calculated molar concentrations of nanocrystals); FIG. 3D is a graph of NIR optical absorbance (at 808 nm) for aqueous suspensions of various concentration FeCo/GC nanocrystals (optical path=1 cm). Solid lines are Beer's law fit for obtaining the molar extinction coefficients.

FIGS. 4A and 4B are graphs showing R₁ (T₁⁻¹) (a) R₂ (T₂⁻¹) (b) vs. metal concentration for the various solutions. Relaxivity values of r₁ and r₂ are obtained from the slopes of the linear fits (solid lines) of experimental T₁⁻¹ and T₂⁻¹ data (designated by symbols) respectively; FIGS. 4C and 4D are MR images of various contrast agents at three metal concentrations; FIG. 4C was generated using a T₂-weighted spin-echo sequence with TE of 60 ms and T_{R} of 3000 ms, while FIG. 4D shows MR images with a T₁-weighted spin-echo sequence with T_{E} of 12 ms and T_{R} of 300 ms. MRI of the FeCo/GC nanocrystal solutions show negative contrast (darkening) in FIG. 4C and positive contrast (brightening) in FIG. 4D

FIG. 5 A-C shows stem cell labeling with functionalized FeCo/GC nanocrystals and demonstration of Near-IR opto-thermal utility, with FIG. 5A showing MRI contrast images of cell pellets (pointed by the arrows) recorded on T₂*-weighted sequences for non-treated control MSCs (mesenchymal stem cells), and FeCo/GC nanocrystal and Feridex® labeled MSCs respectively; FIG. 5B is a bar graph showing cell proliferation assay data over a period of 4 days for nanocrystal labeled MSCs (diagonal stripes, right) and untreated control MSCs (solid, left) and Feridex® labeled MSCs (horizontal stripes, middle); the measured absorbance (at 490 nm) is proportional to the population of live cells; FIG. 5C is a graph temperature evolution curves for solutions of FeCo/GC nanocrystals and Feridex® (all in DMEM cell medium) and cell medium alone (as control) under continuous radiation of a 808 nm NIR laser at 3.5 W/cm²; the heating effect of the FeCo/GC solution is much more significant than the Feridex® and control solutions due to the high NIR absorbance of graphitic shell on the FeCo nanocrystals.

FIG. 6 shows T₁-weighted 3D spoiled gradient recalled echo (SPGR) MR images of a rabbit before (FIG. 6A) and 30 min after (FIG. 6B) initial injection of a solution of ˜4 nm FeCo/GC nanocrystals (metal dose ˜9.6 μM/kg for a ˜5 kg rabbit). The blood pool in the aorta is significantly brightened (positive contrast) in the MRI after injection. Signal increase is also seen in the kidney medulla and cortex due to the high blood volume within the kidney. Little signal enhancement is seen in the muscle. FIG. 6C shows a T₁-weighted image of a diseased rabbit with an aortic stenosis. The narrowing of the blood vessel can be clearly seen as a gap in the bright (white) region along the aorta at the center of the figure. This image was obtained with the injection of a solution of ˜7 nm FeCo/GC nanocrystals (metal dose ˜9.6 μM/kg for a ˜5 kg rabbit) approximately 1 hour after the injection.

FIG. 7 shows mice tumor images obtained using the present ˜7 nm FeCo/GC nanoparticles. FIG. 7A on the left shows an T₂-weighted fast-spin-echo image of a tumor bearing mouse before contrast injection; 7B on the right shows the image of the tumor approximately 24 hours after the injection. Apparent signal decrease due to contrast uptake can be observed; FIG. 7C shows a high resolution T₁-weighted image showing vascularization around the tumor. The image was obtained with a spectral-spatial excitation for fat suppression with a 3D stack-of-spiral SPGR sequence.

FIG. 8 is a pair of graphs showing reduction in tumor volume in experimental mice treated with paclitaxel (8A) and doxorubicin (8C) conjugated to solubilized metal-graphite nanocrystals of the present invention.

FIG. 9 shows high resolution vascular images of a rabbit hind limb imaged with the present metal graphite nanocrystals (9A) and commercially available dextran-coated iron oxide nanoparticles.

FIG. 10 is a graph showing theoretical calculations of a 3D SPGR signal enhancement for blood, comparing the present metal-graphite nanocrystals to other contrast agents.

FIG. 11 is a series of MRI images comparing signal change 24 hours after injection between commercially available Combidex (11A) and 7 nm FeCo/GC functionalized particles (11B). The FeCo/GC functionalized nanoparticles show significant decrease in signal intensity.

### DETAILED DESCRIPTION OF THE PREFERRED EMBODIMENT

### Definitions

The term “Metal nanocrystal” means a particle of less than 20 nm, preferably 1-10 nm nominal diameter, comprising one or more metals. If more than one metal is present, the metals may be alloyed or adjacent, e.g., core-shell configuration. The nanocrystals are preferably sized so that the metal exhibits superparamagnetic behavior, which is a function of both size and atomic properties. Therefore, such metals may be selected from Sub-Group Ib: Transition Metal Elements, Copper, Silver, and Gold; Sub-Group IIa: The Alkaline Earth Metals Beryllium, Magnesium Calcium, Strontium, Barium, Radium; Sub-Group IIb: Transition Metal Elements Zinc, Cadmium, and Mercury; Sub-Group IIIa: Transition Metal Elements, Scandium, Yttrium, Lanthanum; Lanthanides: Cerium, Praeseodymium, Neodymium, Promethium, Samarium, Europium, Gadolinium, Terbium, Dysprosium, Holmium, Erbium, Thulium, Ytterbium, Lutecium; Actinides: Actinium, Neptunium, Plutonium, Americium, Curium, Berkelium; Sub-Group IIIb: Main Group Elements: Boron, Aluminium, Gallium, Indium, Thallium; Subgroup IVa: Titanium, Zirconium, Hafnium, Thorium; subgroup IVb: Germanium, Tin, Lead; Group IV elements: Vanadium, Niobium, Tantalum, Protactinium, Sub-Group Vb: Main Group Elements Arsenic, Antimony, Bismuth; Group VI elements: Chromium, Molybdenum, Tungsten, Uranium, Selenium, Tellurium, Polonium; Sub-Group VIIa: Transition Metal Elements: Manganese, Technetium, Rhenium; and Group VIII elements: Iron, Cobalt, Nickel, Ruthenium, Rhodium, Palladium, Osmium, Iridium, and Platinum. Further details are set forth in http://www.ucc.ie/academic/chem/dolchem/html/elem/group.html. Examples of known metal nanocrystals within this definition include those of Ag, Au, Ir, Rh, Ru, CdSe, ZnSe, PbS, Ag₂S, CdS, Fe₃O₄, CoFe₂O₄, MnFe₂O₄, BaTiO₃, LaF₃, NaYF₄, YF₃, YbF₃, and Ca₁₀(PO₄)6(OH)₂.

The term “nanocrystal,” as used above, is to be distinguished from “nanoparticle,” in that the latter term refers to a particle of a size less than about 20 nm, without reference to the order of the molecules, whereas the present metal nanocrystals have ordered atoms. For example, as reported in Seun et al., “Monodisperse FePt Nanoparticles and Ferromagnetic FePt Nanocrystal Superlattices,” Science 287: 1989-1992 (2000), thermal annealing converts the internal particle structure of FePt nanoparticles from a chemically disordered face-centered cubic phase to the chemically ordered face-centered tetragonal phase and transforms the nanoparticle superlattices into ferromagnetic nanocrystal assemblies. A nanocrystal is typically a single crystal in which the crystal lattice of the entire sample is continuous and unbroken to the edges of the sample, with no grain boundaries.

The term “polar lipid” means a molecule having an aliphatic carbon chain with a terminal polar group. Preferred polar lipids include but are not limited to acyl carnitine, acylated carnitine, sphingosine, ceramide, phosphatidyl choline, phosphatidyl glycerol, phosphatidyl ethanolamine, phosphatidyl inositol, phosphatidyl serine, cardiolipin and phosphatidic acid. Further polar lipids are exemplified in U.S. Pat. No. 6,339,060, “Conjugate of biologically active compound and polar lipid conjugated to a microparticle for biological targeting,” to Yatvin, et al., hereby incorporated by reference.

The term “phospholipid” means a molecule having an aliphatic carbon chain with a terminal phosphate group. Typically the phospholipids will comprise a glycerol backbone, attached to two fatty acid (aliphatic groups) esters and an alkyl phosphate. Suitable phospholipids for use in this invention include, without limitation, dimyristoyl phosphatidylcholine, distearoyl phosphatidylcholine, dilinoleoyl-phosphatidylcholine (DLL-PC), dipalmitoyl-phosphatidylcholine (DPPC), soy phophatidylchloine (Soy-PC or PCs) and egg phosphatidycholine (Egg-PC or PCE). Suitable phospholipids also include, without limitation, dipalmitoyl phosphatidylcholine, phosphatidyl choline, or a mixture thereof. Exemplified below are 1,2-dipalmitoyl-sn-glycero-3 phosphoethanolamine phospholipid and 1,2-Distearoyl-sn-Glycero-3-Phosphoethanolamine.

The term “aliphatic,” as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl” and the like. Furthermore, as used herein, the terms “alkyl,” “alkenyl,” “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

The aliphatic (lipid) alkyl groups employed in the lipids of the invention preferably contain 4-20, more preferably 10-20 aliphatic carbon atoms. In certain other embodiments, the lower alkyl, (including alkenyl, and alkynyl) groups employed in the invention contain 1-10 aliphatic carbon atoms. Illustrative aliphatic groups thus include, but are not limited to, for example, methyl, ethyl, n-propyl, isopropyl, cyclopropyl, —CH₂-cyclopropyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl-n, hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like. The aliphatic groups are hydrophobic and adsorb to the hydrophobic nanoparticle.

The term “alkoxy” (or “alkyloxy”), or “thioalkyl” as used herein means an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” means a group having the structure —NHR′ wherein R′ is alkyl, as defined herein. The term “dialkylamino” means a group having the structure —N(R′)₂, wherein R′ is alkyl, as defined herein. The term “aminoalkyl” means a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

The term “graphitic” means graphite, a crystalline form of the element carbon consisting of parallel layers of hexagonally arranged carbon atoms in planar condensed ring systems. In graphite, each carbon atom is bonded to three other carbon atoms in each layer. After forming a strong covalent sigma (Σ) bond with each neighbor, each carbon atom still has one free electron remaining and these are paired up in a system of weak pi (π) bonds. In graphite, each carbon atom is bonded to three other carbon atoms in each layer. After forming a strong covalent sigma (Σ) bond with each neighbor, each carbon atom still has one free electron remaining and these are paired up in a system of weak pi (Π) bonds. Synthetic graphite such as HOPG (highly oriented pyrolytic graphite) can be intercalated with metal halides, other electron-acceptors or electron-donors. Known materials for inclusion with graphite (doping) include lithium and boron. Suitable electron-acceptor metal halides include ferric chloride (FeCl₃), copper chloride (CuCl₂), aluminum chloride (AlCl₃), nickel chloride (NiCl₂), antimony chloride (SbCl₃), antimony pentachloride (SbCl₅), palladium chloride (PdCl₂), indium chloride (InCl₃), molybdenum chloride (MoCl₅), zirconium chloride (ZrCl₄), tantalum chloride (TaCl₅) and tungsten chloride (WCl₆). See “Intercalation of small graphite flakes with a metal halide, U.S. Pat. No. 4,604,276, hereby incorporated by reference. Graphite may also be fluorinated at high temperatures. The property of a material as “graphitic” may be determined by Raman spectroscopy as described below and elsewhere in the literature. (See Goresy et al., “In situ discovery of shock-induced graphite-diamond phase transition in gneisses from the Ries Crater, Germany,” American Mineralogist, Volume 86, pages 611-621, 2001).

The term “organic molecule” means a molecule suitable for applying to an outer carbon shell of a nanoparticle. Such a molecule may include an aliphatic group, an alkoxy, thioalkyl, alkyamino, or an aromatic group, and substituted variations of such molecules.

The term “organic amphiphilic molecule” means an amphiphile containing a hydrophobic portion, such as an alkyl group of at least 3 carbon atoms linked to a hydrophilic portion, for stabilizing the molecule in aqueous solution. The alkyl group may be a lipid attached to a polar head group, which itself is hydrophilic or is bonded to a hydrophilic polymer. The hydrophilic polymer is preferably a polymer such as PEG. Other hydrophilic polymers suitable for use with the present nanoparticles include branched glycerol and dextran polymers. Water soluble hydrophilic dextran polymers, CAS number 9004-54-0, are available commercially, e.g., from Sigma Aldrich. These range in molecular weight from about 1,000 to about 670,000 D. Polyglycerols (e.g., e.g., poly-2-methyl-2-oxazoline, poly-2-ethyl-2-oxazoline) are further described in U.S. Pat. No. 7,056,532 to Kabanov, et al. issued Jun. 6, 2006, entitled “Compositions for delivery of biological agents and methods for the preparation thereof.” Polyglycerols may also be used as described in U.S. Pat. No. 5,731,006, to Akiyama, et al., issued Mar. 24, 1998, entitled “Gastrointestinal mucosa-adherent granules, pharmaceutical preparations and a coating composition.” In this case, one may use polyglycerol fatty acid esters include, for example, behenyl hexa(tetra)glyceride (e.g., Riken Vitamin Co., Ltd., Japan; Poem J-46B, etc.), stearyl penta(tetra)glyceride (e.g., Sakamoto Yakuhin Kogyo Co., Ltd., Japan; PS-310), stearyl mono(tetra)glyceride (e.g., Sakamoto Yakuhin Kogyo Co. Ltd., Japan; MS-310), stearyl penta(hexa)glyceride (e.g., Sakamoto Yakuhin Kogyo Co., Ltd., Japan; PS-500), stearyl sesqui(hexa) glyceride (e.g., Sakamoto Yakuhin Kogyo Co., Ltd., Japan; SS-500) and stearyl mono(deca)glyceride, as well as mixtures thereof.

The term “NIR” means the near infrared region of the electromagnetic spectrum (from 0.75 to 3 μM).

The term “PEG” means polyethylene glycol, a polymer with the structure (—CH₂CH₂O—)ₙ that is synthesized normally by ring opening polymerization of ethylene oxide. The PEG used herein will impart water (and serum) solubility to the hydrophobic nanoparticle and may be linked to a lipid portion of a polar lipid. The polymer is usually linear at molecular weights (MWs)<10 kD. The PEG used here will have an MW below about 12,000, or 5,400, or 2,000, or below about 300 repeating ethylene oxide units. It is preferred to use higher MW PEGs (higher “n” repeating units) with some degree of branching. Polyethylene glycols of different MWs have already been used in pharmaceutical products for different reasons (e.g., increase in solubility of drugs). Therefore, from the regulatory standpoint, they are very attractive for further development as drug or protein carriers.

For coupling proteins to PEG, usually monomethoxy PEG [CH₃ (—O—CH₂—CH₂)ₙ—OH] is first activated by means of cyanuric chloride, 1,1′-carbonyldiimidazole, phenylchloroformate, or succidinimidyl active ester before the addition of the protein. In most cases, the activating agent acts as a linker between PEG and the protein, and several PEG molecules may be attached to one molecule of protein. The pharmacokinetics and pharmacodynamics of the present nanotubes-PEG-protein conjugates are expected to be somewhat dependent on the MW of the PEG used for conjugation. Generally the presently used PEG will have a molecular weight of approximately 100-2,000 Daltons, or a higher MW of 5,000-16,000 Daltons. Branched PEG up to 50,000 Daltons may be used.

The present PEG may also modified PEG such as PolyPEG® (Warwick Effect Polymers, Ltd., Coventry, England) is new range of materials suitable for the attachment of polyethylene glycol (PEG) to therapeutic proteins or small molecules. These are prepared using Warwick Effect Polymers' polymerization technology, (See U.S. Pat. No. 6,310,149) and contain terminal groups suitable for conjugation with, among other things, lysine, terminal amino and cysteine residues.

The term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of caspase-mediated disorders, as described generally above.

The term “stable” means a solution or suspension in a fluid phase wherein solid components (i.e., nanotubes and drugs) possess stability against aggregation sufficient to allow manufacture and delivery to a cell and which maintain the integrity of the compound for a sufficient period of time to be detected and preferably for a sufficient period of time to be useful for the purposes detailed herein.

The term “superparamagnetic” means particles containing particles of a magnetic material, guaranteeing, after removal of the magnetic field, the absence of any remnant magnetization. That is, for a grain of sufficiently small size, remanence and coercivity go to zero. Superparamagnetism generally occurs when the material is composed of very small crystallites (1-10 nm). Making the particle small enough releases magnetic moments from their constraints, permitting the magnetization of the single-domain particle to fluctuate between the two easy-axis orientations, as in an ideal paramagnet. Superparamagnetism differs from conventional paramagnetism because the effective moment of the particle is the sum of its ionic particles, which can be several thousand spins in a ferromagnetic particle small enough to show superparamagnetism. “Ferromagnetism” refers to common magnetism as exhibited by magnets and magnetic materials. Very fine ferromagnetic particles have very short relaxation times even at room temperature and behave superparamagnetically; that is, their behavior is paramagnetic but their magnetization values are typical of ferromagnetic substances. The individual particles have normal ferromagnetic moments but very short relaxation times so that they can rapidly follow directional changes of an applied field and, on removal of the field, do not hold any remnant moment. Examples of ferromagnetic metals include pure iron, nickel metal, gadolinium, silicon steel, iron-nickel alloys, iron-cobalt alloys, iron-aluminum alloys, and iron-cobalt-nickel alloys.

The term “aromatic molecule” means an aromatic molecule which functions as an “active agent” in a defined environment such as a biological environment, more particularly, within a cell. The aromatic molecule is preferably a biologically relevant molecule including DNA, RNA, proteins, peptide, polypeptide or polynuceotide or a small molecule that normally has poor cellular uptake by itself, such as the exemplified doxorubicin, daunorubicin, fluorescein, or paclitaxel. Cellular uptake is measured by intracellular concentration in target cells or organs, e.g., by immunofluoresence, confocal microscopy or flow cytometry or radio imaging. The term “aromatic” is used in a conventional sense to mean a compound that has special stability and properties due to a closed loop of electrons. The compound (including hetero-aromatic structures) has a planar ring with 4n+2 pi-electrons where n is a non-negative integer (Hückel's Rule). The prototypical aromatic molecule is benzene, but the present aromatic molecules include fused ring structures, and heterocyclic rings such as pyridines, pyrimidines, and pyrazines, which are frequently used in drugs. The term “aromatic small molecule” means a molecule such as those exemplified, and may include nucleic acids or polypeptides in oligomeric (e.g., less then about 4 residue) form, but excludes polymers such as RNA, DNA or polypeptides, where the nanoparticle and the molecule may be simply entangled.

Aromatic small molecule drugs include the exemplified doxorubicin, dauonorubicin and taxol, as well as a wide variety of other drugs such as antibiotics ciprofloxacin, telithromycin (Ketek, Aventis Pharmaceuticals), tetracycline (which is also an MMP inhibitor) and doxycycline. As further examples, the most common drugs used against malaria and as HIV integrase inhibitors including the anti-malarial chloroquine, are based on quinoline, a heteroaromatic compound (similar to naphthalene, except with one carbon replaced by nitrogen). Morphine and other morphanans, based on fused ring aromatic structures, are also included in the present definition. Various nucleic acid analogs also contain aromatic structures, such as the pro-drug 5-fluorocytosine (5-FC) used in cancer therapy. The present complexes are well suited for use with small aromatic ligand molecules that bind to DNA double helical structures by (i) intercalating between stacked base pairs thereby distorting the DNA backbone conformation and interfering with DNA-protein interaction or (ii) the minor groove binders. Both work through non-covalent interaction.

The present aromatic molecules will contain an aryl group.

The term “functionalization” refers to the addition of a solubilizing material, namely the hydrophilic polymer to the graphitic shell of the present metal-graphite nanocrystals. The hydrophilic polymer is tightly linked to the shell, either covalently or by hydrophobic interaction with a hydrophobic portion grafted onto the hydrophilic polymer. In certain embodiments, the hydrophilic polymer is PEG which may be from about 10 to 500 or up to about 1000 PEO (polyethylene oxide) units, and may be straight chain or branched. In certain embodiments, the PEG is coupled to a phospholipid, and for that reason is amine-terminated. It may be referred to as is comprised in an organic amphiphilic molecule, i.e., a lipid or aliphatic (hydrophobic) portion and a hydrophilic portion. The lipid portion may comprise a polar lipid, e.g., a phospholipid. Branched hydrophilic polymers are preferred, and are shown to improve in vivo circulation. Branches may be linked at a single point of juncture, as in a + shape, so that three active agents are linked to the polymer, with the fourth arm linked to the nanoparticle.

The PEG or other hydrophilic polymer may be linked at an end opposite from the amine coupling end to a further molecule, which may be an active agent, a label or a targeting agent for homing the complex to a particular cell type. In certain embodiments, the targeting agent is an antibody, peptide, or other ligand specific for a cell or cell type to be targeted.

### I. INTRODUCTION

The present invention provides magnetic nanocrystals having properties that make them applicable for use, e.g., in MRI, diagnosis and therapy. The graphite coating protects the metal core from chemical reactions and property degradations. It also prevents the metal from leaking out of the coating shell, thus preventing any potential toxicity of the metal. This enables many types of metal nanocrystals to become biocompatible for in vitro and in vivo applications.

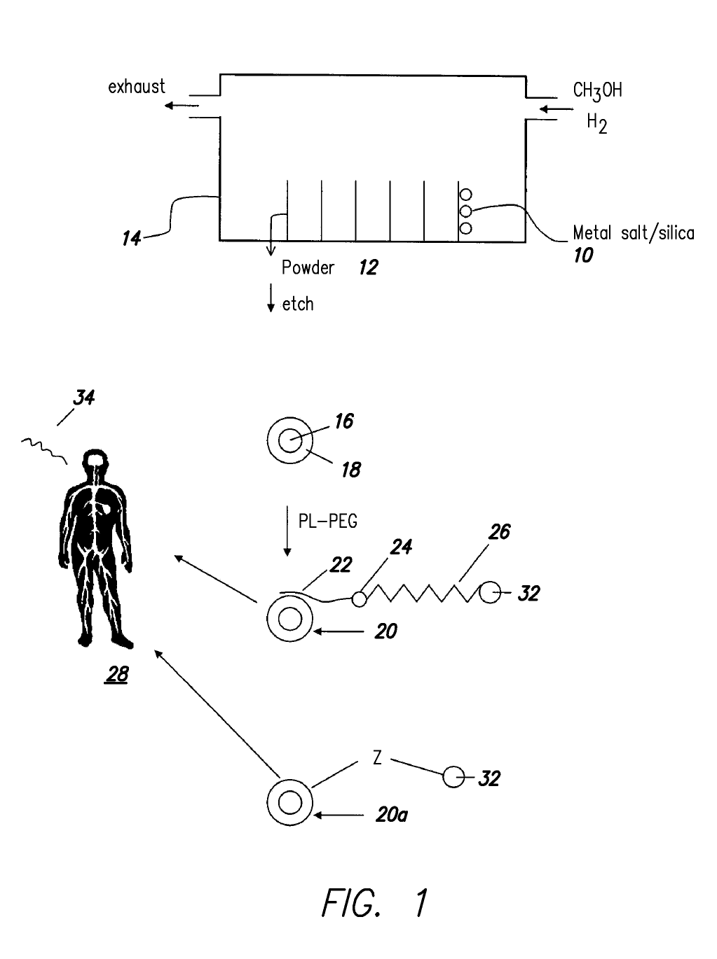

FIG. 1 shows a schematic diagram of the present synthetic methods and resultant materials. The metal(s) to be included in the nanocrystal are dispersed onto silica by impregnation, as described below. The metal salt/silica powder 10 is placed in a CVD furnace 14. Next, the nanomaterial is formed by chemical vapor deposition using methane. Methane CVD is further described in general in Kong et al., “Chemical vapor deposition of methane for single-walled carbon nanotubes,” Chem. Phys. Lett 292:567-574 (1998). In the present method, rather than obtaining nanotubes, a lower level of carbon is provided to provide a graphitic layer, and metals to be included in the core are adsorbed in the form of salts. The salt may, for example, include a chloride, sulfate or nitrate material. After heating and treatment with CH₃OH and H₂, the powder 12 is removed from the CVD furnace 14 and etched to yield a composition of free particles of metal nanocrystal having a graphitic shell 18 directly on the metal, i.e., without an intervening layer, and without any significant oxidation. In a further aspect of the present invention, the metal-graphitic particle is treated to attach a solubilizing layer, e.g., an organic amphiphilic molecule such as phospholipid-polyethylene glycol to produce a particle 20 having a hydrophobic portion (lipid) 22 adsorbed onto the graphitic shell, a linking group (e.g., phosphate) 24, and a hydrophilic tail 26 (e.g., PEG).

As shown at 28, either undecorated, coated particles 16/18, or, preferably, the particles functionalized with PL-PEG can be administered to human or animal subjects, and used for various imaging procedures. In addition, a payload can be attached to the PL-PEG for various therapeutic or diagnostic purposes. Radiation 34 may be applied to the subject in the form of magnetic infrared, x-ray, or other forms, and the resolution of organs or tissues where the present nanoparticles are found will be enhanced.

As an alternative, the FeCo nanoparticle 20a, as shown in FIG. 1, may be covalently functionalized to deliver a payload 32. The linking group, “Z,” may be carboxy, amino, thiol (which may be prepared as disulfide linkages), etc. It will be linked to a carbon atom in the carbon shell and to an organic molecule extending from the carbon shell. Covalent functionalization of the graphitic shell may be carried out by methods known for the covalent functionalization of carbon nanotubes, since both carbon nanotubes and graphitic particles are characterized by sp2 bonds. Thus, the graphitic shell may be treated with EDC (ethyldiamino carbodiimide) and an appropriate amine terminated molecule, such as amino-terminated DNA (Dwyer et al., “DNA-functionalized single-walled carbon nanotubes,” 2002 Nanotechnology 13 601-604), or a fluorescein derivative (Kam et al., “Nanotube Molecular Transporters: Internalization of Carbon Nanotube-Protein Conjugates into Mammalian Cells,” J. Am. Chem. Soc., 126 (22), 6850-6851, 2004). Alternatively, the graphitic shell can be treated with an aromatic solvent (e.g., toluene) and heat to provide a carboxylic acid group, which is then reacted with a variety of amine compounds, as described in U.S. Pat. No. 6,187,823 to Haddon, et al., issued Feb. 13, 2001, entitled “Solubilizing single-walled carbon nanotubes by direct reaction with amines and alkylaryl amines.” Alternatively, amino-modified graphitic shells can be prepared according to the method described in Pantarotto et al., “Translocation of bioactive peptides across cell membranes by carbon nanotubes,” Chem. Comm. 2004, 16, 17.

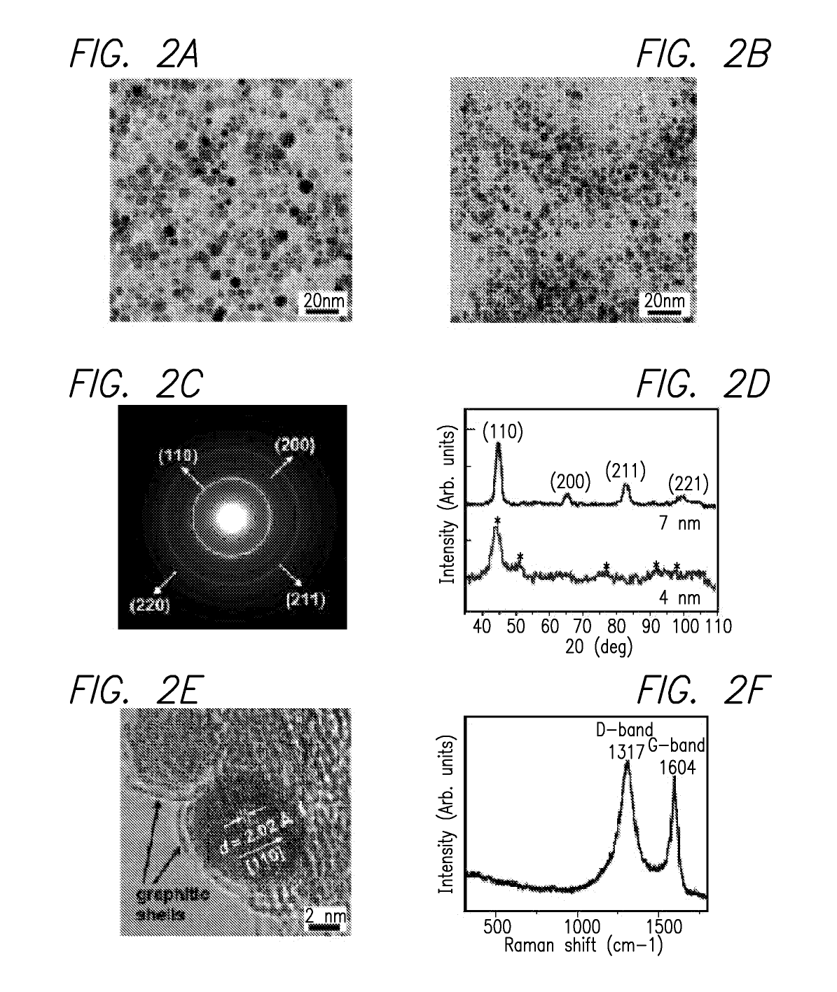

The nanocrystals prepared in the below examples by a chemical vapor deposition (CVD) synthesis of core-shell FeCo nanocrystals had either a single-layer or two layers of graphitic-carbon shell, as shown in FIG. 2E. The nanocrystals exhibit the highest saturation magnetization, high optical absorbance in the near-infrared (NIR) by the graphitic shell, and remarkable chemical stability. The FeCo/graphitic carbon (FeCo/GC) nanocrystals were also functionalized by phospholipid-polyethylene glycol for water solubility and enabling biological applications of FeCo. Multi-functionality is demonstrated including non-toxic labeling of stem cells as contrast agent for magnetic resonance imaging (MRI) with unprecedented high relaxivities and NIR agents for opto-thermal manipulation. Further, the FeCo/GC nanocrystals afford positive contrast enhancement for T₁-weighted MRI owing to unusually high r₁ relaxivities. Thus, advanced MRI can be achieved with lower concentrations than previous contrast agents. The nanocrystals with integrated magnetic and optical properties shall also enable new possibilities in biological imaging, manipulation and therapy.

The present methods are believed to represent the first synthesis of a metal nanocrystal (non-oxidated) in a form that is biocompatible. By using thin graphite carbon shells to cover the metal nanocrystal, toxicity or oxidation is avoided. It also allows for easy functionalization to make it soluble in water. The non-toxic nature and water solubility is essential for biocompatibility while the non-oxidated form and the fact that the carbon shells are thin allows superior relaxation properties compared to other materials. The present materials can replace commercial T₁ (Magnevist® gadopentetate dimeglumine, Berlex Laboratories, Inc.) and T₂ (Feridex® ferumoxides injectable solution, Advanced Magnetics, Inc.) or Combidex® (ferumoxtran-10, Advanced Magnetics, Inc.) MRI contrast agents due to their superior magnetic properties. Combining therapy with visualization is also a feature. Currently, there are no means to do this without invasive procedures such as surgery or catheterization.

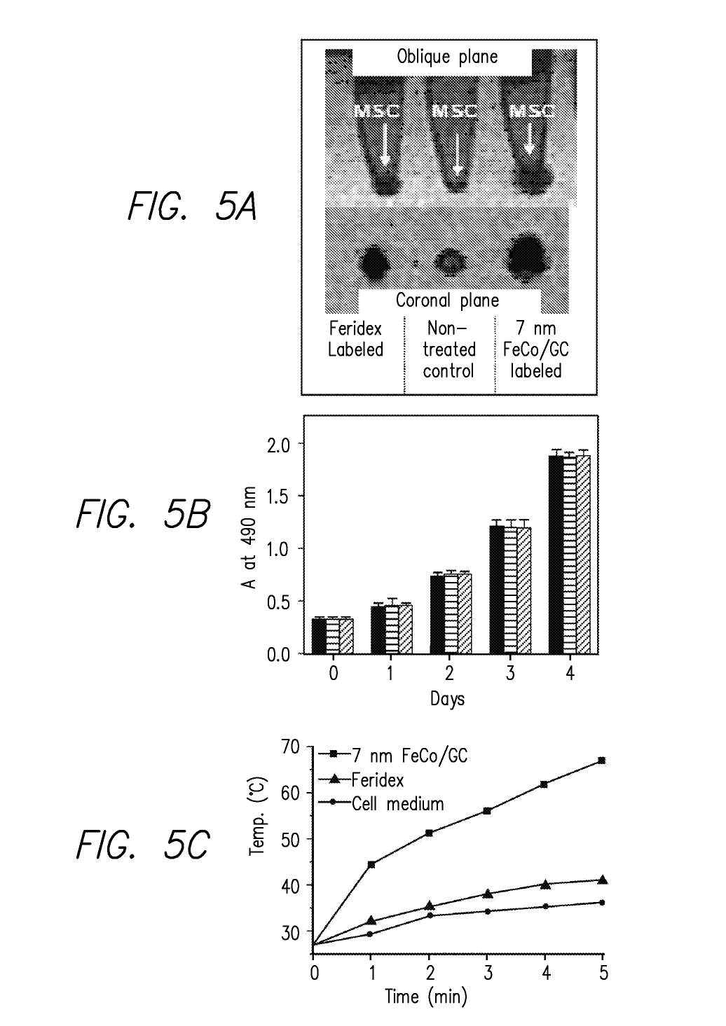

The present synthesis of core-shell FeCo nanocrystals results in an approximately 1- or 2-atom layer shell of graphitic-carbon. This thin shell is sufficient for prevention of oxidation of FeCo, and minimizes the distance for magnetic coupling with spins in the surrounding solution. These particles exhibit high magnetization and high optical absorbance in the near-infrared. By overcoming obstacles in oxide-free synthesis, functionalization and toxicity, the present particles are made highly suitable for in vivo applications. These applications include imaging. The present particles exhibit high r₂ relaxivity, with superior T₂ weighting (darkening). Results have been demonstrated for stem cell labeling, as described below in connection with FIG. 5. The present materials also exhibit positive-contrast enhancement (or brightening) of MRI, owing to high r₁. In addition, lower doses of the present materials are needed, compared to existing agents, in order to achieve bright images in MRI ex vivo and in vivo.

There are many possible variations and modifications of the methods described experimentally below. The sizes of nanocrystals can be modified. This is important for achieving specific relaxation properties in conjunction with specific biodistribution since particle size changes both the relaxation properties and the biological distribution. Sizes can vary between 2 nm to tens of nanometers.

The stability and distribution of the particle in vivo can also be modified by changing the functional materials, specifically the length of PEG and the functional groups of functional materials. Modifying the functional material can be aimed towards increasing circulation time without being taken up by a specific organ or targeting specific disease areas such as cancer or atherosclerosis. Various covalent and noncovalent functionalization schemes can be developed for the graphitic shell on the FeCo core.

### II. SYNTHESIS OF METAL GRAPHITE NANOCRYSTALS

Fe and Co species are first loaded onto high surface area silica powder by impregnation in a methanol solution of mixed Fe and Co salts. The metal loaded silica was dried and heated to 800° C. under H₂ and then subjected to methane CVD for carbon deposition on FeCo nano-alloy formed on silica. Once cooled to room temperature, the material was etched in HF to dissolve the silica support and the resulting pure FeCo/GC nanocrystals were collected.

For ˜7 nm FeCo/GC nanocrystals, 1.00 g of fumed silica (Degussa) was impregnated with 0.145 g of Fe(NO₃)₃.9H₂O and 0.105 g of Co(NO₃)₂.6H₂O in 50 mL methanol and sonicated for 1 h. For ˜4 nm FeCo/GC nanocrystals, 4 times lower metal loading was employed. After removal of methanol and drying at 80° C., we ground the powder and used typically 0.50 g for methane CVD in a tube furnace. The sample was heated in a H2 flow to reach 800° C. and then subjected to a methane flow of 400 cm3/min for 5 min. Upon cooling, we etched the sample with 10% HF in H₂O (80%) and ethanol (10%) to dissolve silica. The FeCo/GC nanocrystals were collected by centrifugation and thoroughly washed. Other alloys (e.g. FeRu) may be prepared in similar processes.

### III. CHARACTERIZATION OF NANOCRYSTALS BY TEM

Transmission electron microscopy (TEM) revealed that the average size of the nanocrystals was tunable by varying the metal loading on silica (see FIG. 2A and FIG. 2B for 7±1.2 nm and 4±0.8 nm nanocrystals respectively, with the mean and standard deviation of sizes measured by TEM for ˜220 nanocrystals in each sample). Crystalline bcc (body-centered cubic)-FeCo core was identified for the 7 nm nanocrystals by electron diffraction (FIG. 2C) and powder X-ray diffraction (XRD, FIG. 2D). The powder XRD data for ˜7 nm and ˜4 nm nanocrystals respectively showed small peaks marked by * on the XRD curve of ˜4 nm nanocrystals, which are assigned to fcc-Co.

High resolution TEM (FIG. 2E) clearly observed the lattice fringes of the FeCo core (d spacing=2.02 A) and graphitic shell (pointed by arrows in FIG. 2E). We observed 1-layer and 2-layers of graphitic shells over-coating the core on 90% and 10% of ˜80 nanocrystals examined respectively. Raman spectroscopy was used to identify graphitic carbon G peak at ˜1600 cm⁻¹ and disordered D peak at ˜1300 cm⁻¹ (FIG. 2F). This Raman spectrum (excitation 785 nm) of ˜7 nm FeCo/GC nanocrystals showing the G and D band of graphitic carbon, provides evidence for graphitic shell.20,21 For 4 nm nanocrystals, mixed bcc-FeCo and fcc(face-centered cubic)-Co phases (see XRD data in FIG. 2D) were identified, indicating phase separation in ultra-small FeCo nanocrystals. We also determined the stoichiometry of our nanocrystals by calcination/burning of the graphitic shells at 500° C., dissolving the metal species in an HCl solution, and measuring the Fe and Co concentrations based on UV-vis absorbance of Fe₃+ and CO₂+. By so doing, we found Fe:Co.40:60 in 7 nm and Fe:Co.12:88 in 4 nm nanocrystals.

We suggest that the FeCo/GC nanocrystal formation involves first FeCo alloying during heating in H₂ and subsequent graphitic shell formation during methane CVD. No metal-carbide phase is observed in the core, which suggests that the deposited carbon mainly interacts with the surface of the metal particle, or has precipitated out of the core to form an encapsulating graphitic layer during cooling. Our CVD synthesis here may be viewed as similar in mechanism to that used for single-walled carbon nanotube growth on metal clusters, which occurs via carbon supersaturation and continuous precipitation from the clusters to form tubular graphitic carbon.22 By using a carbon deficient CVD condition (via the use of chemically stable methane at a high flow rate at 800° C.), we obtain single-layered graphitic carbon encapsulating FeCo nanocrystals rather than nanotubes.

The TEM images were of ˜7 nm and ˜4 nm nanocrystals respectively, shown in FIGS. 2A and 2B, and the selected area electron diffraction pattern was of ˜7 nm nanocrystals. Note that the largest nanocrystals we synthesized were ˜10 nm in mean diameter by increasing the metal precursor loading on silica support. The average sizes of ˜4 nm, ˜7 nm and ˜10 nm were all below the superparamagnetic limit of ˜20 nm (this high limit is due to the low magnetic anisotropy of FeCo, see ref. 1-3).

The nanocrystals were characterized by TEM (Philips CM20 operated at 200 kV and CM300FEG/UT operated at 300 kV), XRD (Philips X'Pert Pro diffractometer using Cu-K (x radiation at 45 kV and 40 mA), Raman (Renishaw 1000 micro-Raman spectrometer with a laser excitation of A=785 nm), and SQUID magnetometer (Quantum Design MPMS-XL). UV-vis-NIR spectrometer (Varian Cary 6000i) was used to measure the absorbance of functionalized FeCo/GC nanocrystal aqueous suspensions and determine the stoichiometry and metal contents in the nanocrystals.

### III. MAGNETIZATION AND STABILITY MEASUREMENTS

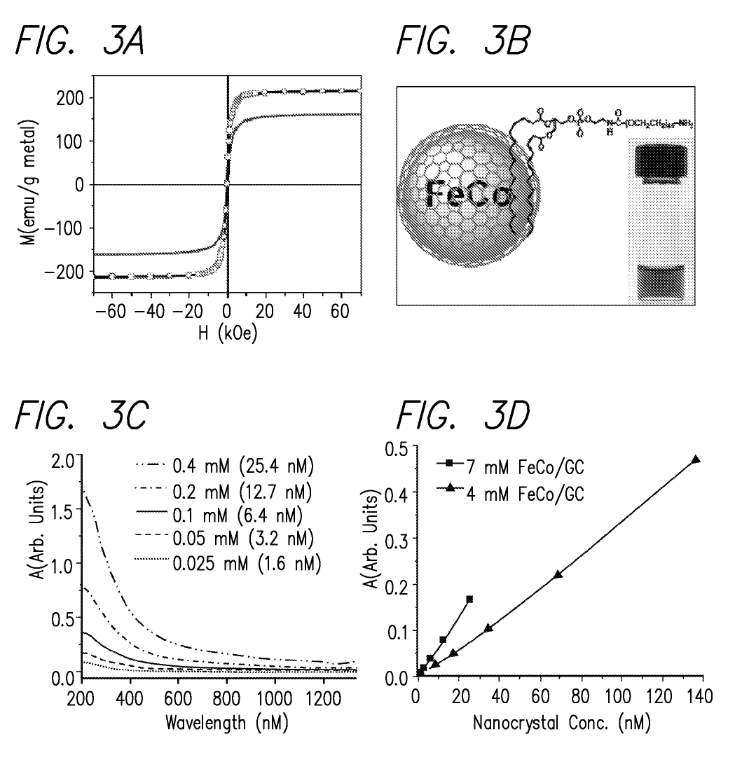

Magnetization measurements by SQUID found that the FeCo/GC nanocrystals were super-paramagnetic at room temperature (FIG. 3A). FIG. 3A shows room temperature magnetization vs. field data for ˜7 nm (line bearing squares) and ˜4 nm (line with no squares) nanocrystals measured shortly after synthesis, and after one month exposure in ambient air for the ˜7 nm nanocrystals (boxes, no degradation from the solid black line). Note that no hysteresis loop exists in the solid lines comprised of data of cycling the field, due to superparamagnetism of the nanocrystals. From the hysteresis loop, a number of primary magnetic properties of a material can be determined, including retentivity, residual magnetism, coercive force (the amount of reverse magnetic field which must be applied to a magnetic material to make the magnetic flux return to zero—the value of H at point c on the hysteresis curve), etc.

By determining the total metal amount in the SQUID sample using the aforementioned calcinations/HCl/UV-vis method, we found that the saturation magnetization (Ms) of the 7 nm FeCo/GC nanocrystals was 215 emu/g (FIG. 3A), close to bulk FeCo (235 emu/g). (The saturation magnetization is the maximum induced magnetic moment that can be obtained in a magnetic field (Hsat); beyond this field no further increase in magnetization occurs.)

The present FeCo/Gc nanocrystals exhibit superior saturation magnetization. It is substantially higher than for SPIO (superparamagnetic iron oxide). This may be seen in reference to R Mendoza-Reséndez et al, “Microstructural characterization of ellipsoidal iron metal nanoparticles,” 2004 Nanotechnology 15 S254-S258. The authors prepared metal particles, which consist of a metal core of α-Fe and an oxide layer about 5 nm thick, with a spinel structure. The magnetic properties of this material showed a saturation magnetization of 160 emu/g. The present nanoparticles exceeded this, showing a highest saturation magnetization of 235 emu/g.

For the ˜4 nm sample, the measured Ms was 162 emu/g (FIG. 3A, lower curve) and the lower Ms was due to the mixed bcc-FeCo and fcc-Co phases in the nanocrystals. Deviation from bulk Ms value for the ˜7 nm sample was partly due to the existence of a few percentage of small nanocrystals (down to 4 nm) in the material. Importantly, the FeCo/GC nanocrystals exhibited superior chemical stability against HF acid etching and oxidation resistant in air, without any degradation in Ms over a monitoring period of 1-month exposure in air. These illustrated the robustness of the single-layered graphitic shell against chemical attacks in gas and liquid phases.

Stoichiometry and metal contents in FeCo/GC nanocrystals was carried out by a calcination/HCl/UV-vis method in which HCl solutions were used to dissolve known amounts of Fe₂O₃ (Aldrich, 99.8%) and CoO (Aldrich, 99.99+%). The resulting solutions were used to calibrate characteristic UV-vis absorbance peaks and molar extinction coefficients of Fe₃+ at 362 nm and CO₂+ at 691 nm respectively. The stoichiometry and metal contents of FeCo/GC nanocrystals were determined by calcinations of the graphitic shells at 500° C. in air, dissolving the metal species in HCl solutions and measuring the UV-vis absorbance at the two wavelengths.

### IV. FUNCTIONALIZATION WITH PL-PEG (PHOSPHOLIPID-POLYETHYLENE GLYCOL)

We obtained stable aqueous suspensions of FeCo/GC nanocrystals by non-covalent functionalization with phospholipid-poly(ethylene glycol) (PL-PEG, MW of PEG=2000) molecules (FIG. 3B). FIG. 3B is a schematic of a FeCo/GC nanocrystal, structure of the phospholipid molecule used for functionalization, and a photograph of a PBS suspension of functionalized FeCo nanocrystals taken after heating to 80° C. for 1 h. The hydrocarbon chains (16-18 carbons) of phospholipids adsorbed onto the graphitic shells via van der Waals and hydrophobic interactions while the hydrophilic PEG chain (45 monomer units) extends into the aqueous phase to impart solubility (FIG. 3B). This result resembled PL-PEG functionalization of carbon nanotubes.^{23,24} The functionalized nanocrystals were highly stable in phosphate buffered saline (PBS) solutions against aggregation for >6 months and even after extended heating at 70-80° C. (photo in FIG. 3B). The nanocrystals were also stable in PBS containing serum proteins without aggregation. Further, no sedimentation of the functionalized nanocrystals was observed under centrifugation at 8,000 g for 30 min, whereas nanocrystals without PL-PEG form an aggregated pellet without any centrifugation. These results confirm that PL-PEG functionalization is robust without any detachment under physiologically relevant conditions. Dynamic light scattering measurements found that the hydrodynamic diameters of functionalized FeCo-GC nanocrystals were ˜30 nm, a reasonable value since the length of a PL-PEG molecule was ˜17 nm when unfolded. Similar hydrodynamic size was measured for nanocrystal solutions after aging for >6 months and after extended heating to 70° C., further indicating no detachment of PL-PEG under these conditions.

### V. ABSORBANCE SPECTRA

The UV-vis-NIR optical absorption spectra of aqueous suspensions of FeCo/GC nanocrystals exhibited monotonic increases in absorbance at shorter wavelengths (FIG. 3C), characteristic of the π-plasmon of graphitic carbon including graphitic shells.25 FIG. 3C shows UV-vis-NIR absorption spectra of PBS suspensions of ˜7 nm functionalized nanocrystals at various molar metal concentrations (values in brackets are calculated molar concentrations of nanocrystals). NIR optical absorbance (at 808 nm) for aqueous suspensions of various concentrations FeCo/GC nanocrystals (optical path=1 cm). Solid lines are Beer's law fit for obtaining the molar extinction coefficients. The top line is for the 7 nm nanocrystals.

Appreciable absorbance in the NIR (between 700 and 1,100 nm) due to the plasmon tail was observed with molar extinction coefficients of ∈≅6.58 μM-1 cm⁻¹ and 3.50 μM-1 cm-1 (based on calculated molar concentration of nanocrystals) at λ=808 nm for 7 nm and 4 nm nanocrystals respectively (FIG. 3D). The corresponding metal-atom (Fe—Co combined) based molar extinction coefficients were 418 M-1 cm-1 and 1,192 M-1 cm-1 respectively.

We investigated the potential utilization of the NIR optical properties of the FeCo/GC nanocrystals. We found that under λ=808 nm NIR laser radiation, an aqueous suspension of FeCo/GC nanocrystals exhibited significant temperature increases over time, while a Feridex suspension showed little heating effect (FIG. 5C) consistent with the negligible optical absorbance of Feridex in the NIR (confirmed by control experiment). To measure NIR laser radiation, solutions of FeCo/GC nanocrystal suspension, Feridex (metal concentration ˜25 μg/mL in both) in DMEM cell medium and cell medium alone (as control) were irradiated by a 808-nm laser at 3.5 W/cm2 fluence. Temperature was measured at 1-min interval with a thermocouple (not directly under the laser beam). The graph in FIG. 5B shows cell proliferation assay data over a period of 4 days for nanocrystal labeled MSCs (right) and untreated control MSCs (left) and Feridex labeled MSCs (middle). The measured absorbance (at 490 nm) is proportional to the population of live cells.

This illustrated the optical absorbance at 808 nm by our FeCo/GC nanocrystals affording conversion of NIR photon energy into thermal energy, similar to gold nanoshells,⁷ nanorods,⁸⁻¹⁰ and carbon nanotubes.²⁴ Since biological molecules are relatively transparent in the NIR optical window, NIR absorbance of nanomaterials can be utilized for intracellular manipulation of living cells including controlled drug release and destruction of cancer cells.^{7,9,10,24}

### VI. MRI CONTRAST AGENTS WITH ULTRAHIGH RELAXIVITIES AND SENSITIVITIES

An important goal of MRI contrast agents has been lowering the required dosage (lower concentration), increasing stability in biological media and achieving positive contrast enhancement. Our functionalized ultra-high relaxivity FeCo/GC nanocrystals achieve this goal. In preliminary animal experiments, we have indeed observed long-lasting (>30 min, vs. only seconds for Gd agents) positive-contrast T₁-weighted intravascular MRI of rabbit using relatively low dose (˜9.6 μM/Kg) of the 4 nm and 7 nm FeCo/GC nanocrystals. Lastly, we obtain magnetic nanocrystals with NIR properties for multi-functions including imaging, diagnosis and therapy. With the FeCo/GC nanocrystals, selective in vivo targeting of tumor sites, high-contrast MRI confirmation of the targeting and NIR laser triggered tumor destruction are envisioned.

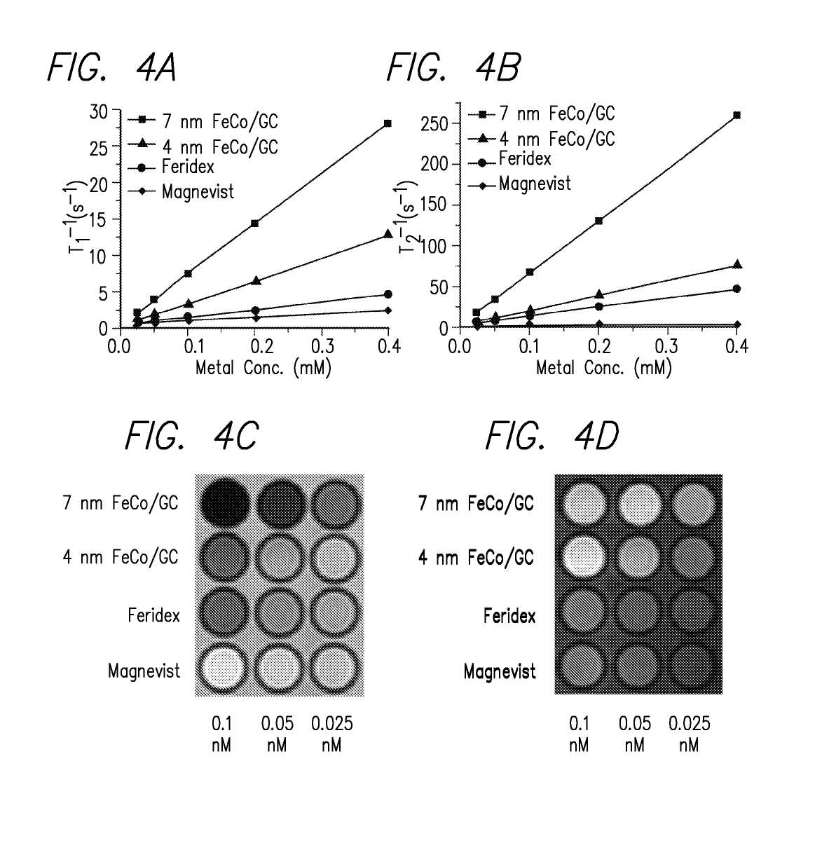

We measured longitudinal (T₁) and transverse (T₂) relaxation times of protons in water solutions of functionalized FeCo/GC nanocrystals in a 1.5 Tesla magnetic field, derived r₁ and r₂ relaxivity (FIG. 4A & B) and compared (Table 1) with commercial MRI contrast agents Feridex²⁶ (with ˜15-21 nm γ-Fe₂O₃ core and dextran coating; Mₛ˜70 emu/g, about 3 fold lower than FeCo) and Magnevist (a gadolinium complex).

TABLE 1

| Sample | r₁ [mM⁻¹s⁻¹] | r₂ [mM⁻¹s⁻¹] | r₂/r₁ |
|---|---|---|---|
| 7 nm FeCo/GC | 70 | 644 | 9.2 |
| 4 nm FeCo/GC | 31 | 185 | 6.0 |
| Feridex | 10 | 104 | 10.4 |
| Magnevist | 4.6 | 4.5 | 1.0 |

The sample above labeled “Feridex” refers to Feridex® ferumoxides injectable solution from Berlex Imaging, used as ˜15-21 nm γ-Fe₂O₃ core particles. “Magnevist” refers to Magenevist® (gadopentetate dimeglumine) imaging agent also from Berlex Imaging, an operating unit of Berlex, Inc.

Table 2 shows that T₁ and T₂ relaxivities—r₁, r₂,—and r₂/r₁ ratios for PL-PEG functionalized FeCo/GC nanocrystals, Feridex, and Magnevist respectively. Both r₁ and r₂ are unusually high compared to conventional Gd and iron oxide based contrast agents, opening new possibilities in advanced positive and negative contrast MRI. The graphs in FIGS. 4A and 4B show T₁⁻¹ and T₂⁻¹ vs. metal concentration for the various solutions. Relaxivity values of r₁ and r₂ are obtained from the slopes of the linear fits (solid lines) of experimental T₁⁻¹ and T₂⁻¹ data (symbols) respectively.

A surprising result was that the 7 nm FeCo/GC nanocrystals exhibited the highest r₂ (644 mM⁻'s⁻¹) among reported values for contrast agents at 1.5 Tesla and afforded superior T₂ negative contrasts for MRI than Feridex (FIG. 4C). The photograph in FIGS. 4C and 4D are MR images of various contrast agents at three metal concentrations generated on a T₂-weighted spin-echo sequence with T_{E} of 60 ms and T_{R} of 3000 ms. FIG. 4D is an MR images with a T₁-weighted spin-echo sequence with T_{E} of 12 ms and T_{R} of 300 ms. Positive contrast (brightening) is seen in the MRI of the FeCo/GC nanocrystal solutions. The present materials also exhibit improved r₁ relaxivity, which is believed to be attributable to the high saturation magnetization and the high aqueous stability of the nanocrystals without aggregation. The relaxivity was found to be independent of the length of hydrophilic polymer (PEG) coating.

The high relaxivity was attributed to high Mₛ of the FeCo core and ultra-thin, single graphitic-shell structure, affording effective magnetic relaxations to the proton spins around the nanocrystals. The 1- and 2-shelled FeCo nanocrystal were novel in both high oxidation resistance and maximum magnetic coupling to surrounding spins. Also important was that our nanocrystals exhibited smaller r₂/r₁ than Feridex and very high r₁, making them useful as T₁ positive contrast agents (FIG. 4D) that were difficult to obtain with oxide nanoparticles.²⁷

Relaxation parameters were compared at 3 T (3 Tesla) and 1.5 T. The r₁ values at 3 T were half the values at 1.5 T, but the r₂ values were the same for both fields. Data is summarized below.

TABLE 2

| | r₁ | r₂ | r₂/r₁ |
|---|---|---|---|
| 1.5 T | | | |
| | 72/(mmol s) | | |
| 7 nm FeCo/GC | 6.3e−15/(NPs) | 670 | 9.31 |
| | 37 | | |
| 4 nm FeCo/GC | 1.85e−16/(NPs) | 180 | 4.86 |
| 3.0 T | | | |
| Feridex | 14 | 180 | 12.86 |
| Combidex | 16 | 100 | 6.25 |
| 7 nm FeCo/GC | 37 | 650 | 17.6 |
| 4 nm FeCo/GC | 19 | 170 | 9.0 |
| Feridex | 6.4 | 190 | 29.7 |
| Combidex | 8.3 | 99 | 11.9 |

Aqueous solutions of various concentrations FeCo/GC nanocrystals were used for T₁ and T₂ measurements using a 1.5 T and 3.0 T GE excite whole body MRI scanners (maximum gradient: 40 mT m⁻¹, maximum slew rate: 150 mT m⁻¹ms⁻¹). For T₁ measurements, inversion recovery sequence was used with a field of view (FOV) of 24 cm, slice thickness of 3 mm and an imaging matrix size of 128 by 128. Pulse repetition time (T_{R}) was 6000 ms and the echo time (T_{E}) was 50 to 350 ms at 50 ms steps. T₂ measurements were performed using spin-echo sequence with a FOV of 24 cm, slice thickness of 3 mm and an imaging matrix size of 192 by 160. T_{R} was 3000 ms and T_{E} was 9, 12, 20, 30, 50, 80, 160 ms. The data was then analyzed to extract T₁ and T₂ values through non-linear least-square fit to the inversion recovery curve and the spin-echo decay curve respectively. For the T₂*-weighted MRI imaging of the MSCs, a gradient recalled echo (GRE) sequence was used with a FOV of 16 cm, slice thickness of 1 mm and an imaging matrix size of 256 by 256. T_{R} was 100 ms and T_{E} was 10 ms.

Circulation times in rabbits were measured for different contrast agents: Magnevist ˜10 min.; Combidex: ˜20 hrs. 7 nm FeCo/GC: ˜4.5 hrs. 4 nm FeCo/GC: ˜5 hrs. The circulation times could be adjusted by surface chemistry. That is, the branching of the PEG, the length of PEG cahins and the number of PEG chains linked to the graphite ether covalently or by adsorption through a phospholipid can be controlled, and this will affect in vivo fate. T₁ and T₂ and relaxivities r₁, r₂ and r₂/r₁ ratios and were also calculated for PL-PEG functionalized nanoparticles at 1.5 T, as indicated in the following table, and compared with compared with commercially available materials:

TABLE 3

| sample | r₁ [mM⁻¹s⁻¹] | r₂ [mM⁻¹s⁻¹] | r₂/r₁ |
|---|---|---|---|
| 10 nm FeCo/GC | 69 | 700 | 10.1 |
| 7 nm FeCo/GC | 67 | 580 | 8.7 |
| 4 nm FeCo/GC | 32 | 200 | 6.3 |
| 4 nm Co/GC | 31 | 230 | 7.4 |
| 3 nm Co/GC | 24 | 170 | 7.1 |
| Feridex (SPIO) | 10 | 104 | 10.4 |
| Combidex (SPIO) | 15 | 97 | 6.5 |
| Magnevist (Gd complex) | 4.6 | 4.5 | 1.0 |

### VII. CELL LABELING

The biological applications of the PL-PEG functionalized FeCo/GC nanocrystals was demonstrated by carrying out magnetic labeling of mesenchymal stem cells (MSCs). Both the 7 nm FeCo/GC nanocrystals and Feridex® iron oxide nanoparticles were found to be spontaneously internalized into MSCs upon 1-day incubation, without the need of any delivery agent. This was evidenced by enhanced contrast in T₂-weighted MR images of cell pellets after the incubation (FIG. 5A). The photograph in FIG. 5A shows MR contrast images of cell pellets (pointed by the arrows) recorded on T₂-weighted sequences for non-treated control MSCs, and FeCo/GC nanocrystal and Feridex (labeled MSCs respectively. The efficient cellular uptake was consistent with facile particulate endocytosis observed with MSCs previously.²⁸ The FeCo/GC-labeled MSCs exhibited significantly higher MRI contrast than Feridex® labeled cells, while UV-vis characterization of the species extracted from the labeled cells showed similar metal amounts in the FeCo/GC labeled cells (9.4±0.3 fmole metal/cell, or 0.54±0.02 pg metal/cell) and Feridex® labeled cells (9.0±0.3 fmole metal/cell, or 0.50±0.02 pg metal/cell). This demonstrated FeCo/GC nanocrystals as high performance MRI contrast agents capable of contrast enhancement at lower doses than existing materials.

To assess the biocompatibility of our FeCo/GC nanocrystals, CellTiter 96 MTS assays were used (Promega) to investigate the proliferation of MSCs with internalized FeCo/GC nanocrystals. Similar cell proliferation behavior was observed over a 4-day monitoring period for untreated control cells, FeCo/GC labeled cells and Feridex® (an FDA approved agent) labeled cells (FIG. 5B). The graph in FIG. 5B shows cell proliferation assay data over a period of 4 days for nanocrystal labeled MSCs (right) and untreated control MSCs (left) and Feridex® labeled MSCs (middle). The measured absorbance (at 490 nm) is proportional to the population of live cells. Various in vitro toxicity assays were also carried out to confirm the lack of cyto-toxicity of the FeCo/GC nanocrystals. In preliminary in-vivo animal experiments, we injected the PL-PEG functionalized nanocrystals (˜4 nm) into a rabbit, and observed no apparent acute toxicity or obvious negative heath problems of the rabbit over a monitoring period of ˜2 month. These results were expected considering the biocompatibility of various water solubilized carbon materials including carbon nanotubes.^{29,30} Due to the tightness of the graphitic shell to molecules in both gas and liquid and the superior chemical stability against shell-opening, toxicity of Co in the core is not expected. The graph in FIG. 5C shows temperature evolution curves for solutions of FeCo/GC nanocrystals and Feridex (all in DMEM cell medium) and cell medium alone (as control) under continuous radiation of a 808 nm NIR laser at 3.5 W/cm². The heating effect of the FeCo/GC solution is much more significant than the Feridex and control solutions due to the high NIR absorbance of graphitic shell on the FeCo nanocrystals.

FeCo/GC nanocrystals were added to a PBS solution of PL-PEG (Avanti Polar Lipids, 1 mg/mL) and sonicated for 1 h. Centrifugation at 10,000 g for 5 min was used to remove any aggregates. Excess PL-PEG was removed by filtration and the remaining nanocrystals with adsorbed PL-PEG exhibited excellent stability in various aqueous solutions.

For MSC culture and MSC labeling by FeCo/GC nanocrystals and Feridex, MSCs were cultured in DMEM supplemented with 10% FBS and 1% penicillin-streptomycin (Invitrogen). Labeling of MSCs with magnetic FeCo/GC nanocrystals and Feridex were carried out in 6-well plates, with the cells pre-seeded for ˜18 h before labeling. PL-PEG functionalized FeCo/GC nanocrystal or Feridex suspensions were added to 2 wells each (˜2×106 cells/well) at a final concentration of 1.2 μmol/mL. Unlabeled MSCs in the other 2 wells were used as controls. The incubations were carried out at 37° C. and in 5% CO₂ atmosphere for 1 day. After incubation, the cell medium was removed from the wells, and the cells were washed and detached from the wells by addition of trypsin-EDTA solution (Invitrogen) for MRI or for proliferation assay.

The assay of cellular metal uptake by a UV-vis method was carried out as follows: After an MRI experiment, the FeCo/GC nanocrystal or Feridex labeled MSCs were collected and calcined at 500° C. in air. After dissolution of the residue in HCl solutions, the amount of metal (Fe and Co in the case of our nanocrystals and Fe in the case of Feridex) was determined by the UV-vis method above.

The cell proliferation assay was carried out as follows. The FeCo/GC nanocrystal or Feridex labeled MSCs were plated in a 6-well plate (˜4×10⁴ cells/well) together with untreated control cells and kept at 37° C. in 5% CO₂. The cell medium was changed to fresh medium daily. CellTiter96 proliferation assay (Promega) was performed at 24 h intervals for 4 days. The CellTiter 96 assay measures dehydrogenase enzyme activity found in metabolically active cells.¹²

### VIII. IN VIVO EXPERIMENT RESULT—IMAGING OF AORTA

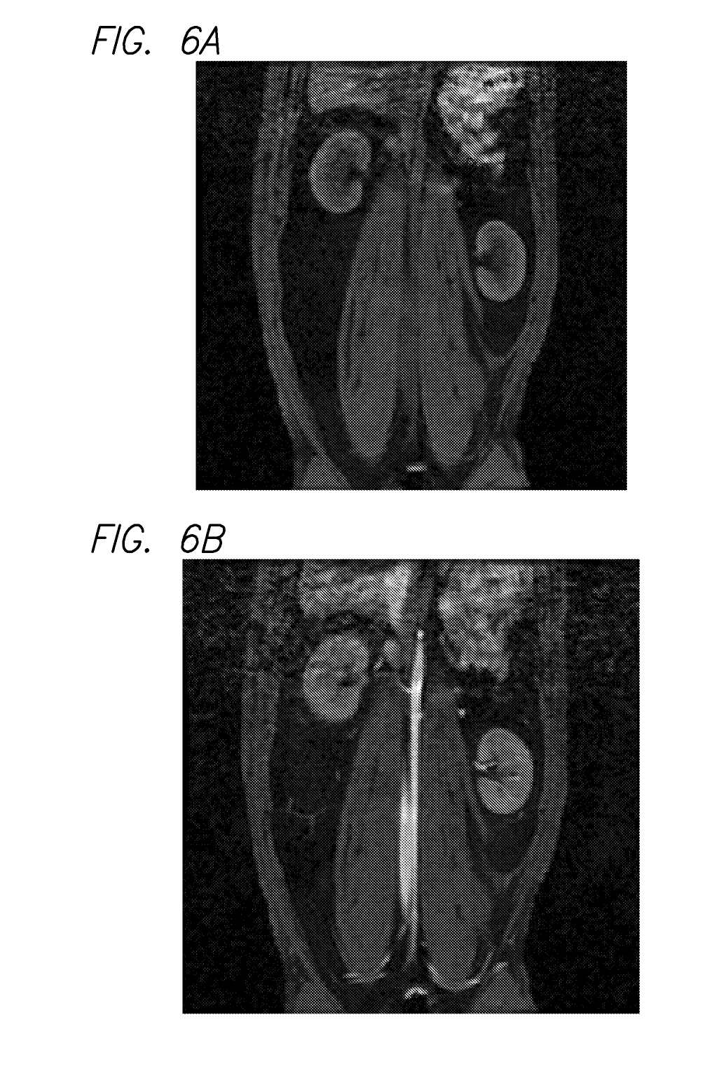

Described below are the results of an in vivo experiment where ˜4 nm PL-PEG coated nanocrystals were injected into a rabbit. Further described are results including in vivo intravascular imaging, and, as shown in FIG. 6b, positive-contrast brightening of the rabbit blood pool, using a metal concentration only 10% of the existing Gd agent. The PL-PEG nanocrystals were stable in blood circulation without aggregation (no PL-PEG desorption in vivo), or leaking out of the blood stream or body in the time scale needed for MRI, and can be used for MRI contrast enhancement for at least 30 min (anesthesia time for the rabbit). Conventional Gd agents only allow for MRI within seconds of injection due to rapid leak-out, and require much higher dose than our nanocrystals. This in vivo result is exciting since it shows the potential of positive contrast MRI with our nanocrystals (a major goal in MRI contrast agent) and the PL-PEG nanocrystals are stable in vivo and remain in blood circulation for long time. More systematic in vivo research is necessary and is currently being conducted.

A ˜4 nm FeCo/GC nanocrystal solution was intravenously injected into a rabbit under anesthesia. 4 mL of 3 mM (total metal molar concentration, determined by the calcination/HCl/UV-vis method above) solution was repeatedly injected four times in 10 min interval. T₁ weighted images were acquired before any injection and after each injection. The total dosage of the four injections was 9.6 μM/kg (the rabbit weight ˜5 kg), which is less than 10% of the recommended dosage of Magnevist injection (100 μM/kg). For T₁ weighted imaging, fat saturated three dimensional (3D) spoiled gradient recalled echo (SPGR) sequence was used with a TR of 33 ms, TE of 4 ms and 45° flip angle. The imaging matrix size was 192 by 160 with a 20 cm FOV and the slice thickness was 1.5 mm with 28 slices. The resulting scan time for the 3D imaging volume was 2 min 54 s.

The photographs of FIG. 6 show a vascular MRI imaging result with the rabbit in-vivo. FIG. 6A shows an MRI of a rabbit before injection of a solution of the PL-PEG coated 4 nm FeCo/GC nanocrystal. FIG. 6B was taken 30 min after injection. The blood in aorta is lightened in this T₁-weighted MRI image, achieving positive contrast with only 10% of metal typically needed for existing Gd agent. Long circulation in blood and long lasting contrast enhancement are also found (>30 min, anesthesia time), which is also superior to Gd agent. Note that very little signal (or FeCo/GC) is seen in the muscle.

Rabbit experiments showed that the particles accumulate in the liver after 12 hours, but start to secrete through the intestine through the biliary system. Fluorescence imaging for labeled particles in mice confirm excretion through feces and urine.

### IX. PREPARATION OF AUFE-GRAPHITIC CORE SHELL NANOPARTICLES

Gold iron alloy nanoparticles about 10 nm in diameter enclosed by graphitic carbon were successfully made by solution chemistry, chemical vapor deposition (CVD) and subsequent purification. High Resolution Transmission Electron Microscopy (HRTEM) and Energy Dispersive X-ray Spectroscopy (EDS) analysis indicated that in each particle, the core is made of AuFe alloy (atomic ratio of Au to Fe is about 2:1, this ratio is tunable) and the shell typically compose of few atomic layers of graphitic carbon. Water suspension of such particles has been found to be a good absorption medium for x-ray/CT imaging and is a good candidate to replace iodine-based contrast agents that are currently used for CT imaging. In addition, it was found that the sample has significantly shorter T₂ relaxation time compared to water and thus can serve as dual contrast agent for MRI as well as x-ray imaging.

The procedure for making AuFe—C (iron gold-carbon shell) nanoparticles was as follows: Fe(NO₃)₂₋₉H₂O+HAuCl₄ was added to fumed-silica. Water was added, and the mixture was stirred for 5 min. and sonicated for 90 min. Then, it was heated on a hot plate overnight, and the remaining solid was ground. Next a CVD process was carried out, as described above, using a hydrocarbon such as methane and heating to 900° C. The resulting material was etched with HF to remove the silica. The resultant material was collected by centrifuge, the remaining solid rinsed with water and resuspended in water. To add a phospholipid coating to the Au—Fe—C particles, the material was added to a solution of phospholipid at 0.1 mg/mL and sonicated for 30 min. until a nanoparticle suspension was obtained. For TEM studies, a drop of suspension was applied on a lacey carbon grid and dried in air. TEM studies showed that the mean size of the AuFe—C particles obtained was 12 nm, the median size was 11 nm, with a standard deviation of 15.6%. The present methods yield particles with a desirable size distribution, in that particles are relatively uniform in size and shape, i.e., within about 20% of the nominal diameter, making the particles well suited for use as contrast agents.

The graphitic shells were observed as layers between one and three atoms thick. The particles exhibited superparamagnetic behavior and CT contrast enhancement. As is known, the CT image pixel itself is displayed according to the mean attenuation of the tissue(s) that it corresponds to on a scale from −1024 to +3071 on the Hounsfield scale (HU). The present materials exhibited HU=280 in suspension, while a similar concentration of commercial iodine contrast agent exhibited and HU=570, and barium contrast agent measured HU=250.

### X. FORMULATIONS FOR IN VIVO USE

As described in connection with Section VIII: “In-Vivo Experiment Result,” the present nanocrystals may be coated with an amphiphilic organic molecule and prepared for injection. As described in Section VIII, FeCo/graphite nanocrystals coated with PL-PEG was prepared as an aqueous solution for injection into a rabbit.

If desired, the contrast agent may further comprise a physiologically compatible suspending or viscosity-increasing agent, which will assist in providing relative uniformity or homogeneity to the contrast agent. A number of such agents are available, including xanthan gum, acacia, agar, alginic acid, aluminum monostearate, bassorin, karaya, gum arabic, unpurified bentonite, purified bentonite, bentonite magma, carbomer 934P, calcium carboxymethylcellulose, sodium carboxymethylcellulose, carboxymethylcellulose sodium 12, carrageenan, cellulose (microcrystalline), dextran, gelatin, guar gum, hydroxyethylcellulose, hydroxypropylcellulose, hydroxypropylmethylcellulose, magnesium aluminum silicate, methylcellulose, pectin, casein, polyethylene oxide, polyvinyl alcohol, povidone, propylene glycol, alginate, silicon dioxide, silicon dioxide colloidal, sodium alginate and other alginates, and tragacanth. As those skilled in the art would recognize, wide ranges of suspending agents can be employed in the contrast agent of the invention, as needed or desired. Preferably, however, the suspending agent is present in an amount of at least about 0.05% by weight, more preferably at least about 0.1% by weight, and generally less than about 1% by weight, more preferably less than about 0.5% by weight.

If the compositions of the invention are used as intravascular agents, osmolarity is important to prevent blood cell damage. It should be approximately the same value as that of human blood. As those skilled in the art will recognize, the osmolarity of a solution may be controlled by regulating the use of osmotically active materials in the contrast agent formulation. Osmotically active materials include such physiologically compatible compounds as monosaccharide sugars or sugar alcohols, disaccharide sugars, amino acids and various synthetic compounds. Suitable monosaccharide sugars or sugar alcohols include, for example, erythrose, threose, ribose, arabinose, xylose, lyxose, allose, altrose, glucose, mannose, idose, galactose, talose, ribulose, fructose, sorbitol, mannitol and sedoheptulose, with preferable monosaccharides being fructose, mannose, xylose, arabinose, mannitol and sorbitol. Suitable disaccharide sugars include, for example, lactose, sucrose, maltose, and cellobiose. Suitable amino acids include, for example, glycine, serine, threonine, cysteine, tyrosine, asparagine, glutamine, aspartic acid, glutamic acid, lysine, arginine and histidine. Synthetic compounds include, for example, propylene glycol, polypropylene glycol, ethylene glycol, polyethylene glycol and polyvinylpyrrolidone.

Typically, to achieve the preferred ranges of osmolarity in the contrast agent of the invention, less than about 25 g/l, more preferably less than about 20 g/l, even more preferably less than about 15 g/l, and most preferably less than about 10 g/l of the osmotically active materials are employed, and in some instances no osmotically active material is used. A most preferred range of osmotically active material is between about 1 and 10 g/l.

Although the most desirable pH for the contrast agent of the present invention may vary, as those skilled in the art will recognize, the preferred pH range for most diagnostic uses is generally between about 3 and about 10 pH units, more preferably between about 5 and 8 pH units. The desired pH can be achieved and maintained through the use of physiologically compatible pH regulating additives such as suitable bases, buffers and the like, as one skilled in the art will recognize. Particularly preferred buffers include sodium acetate and glacial acetic acid buffer.

Other procedures for preparation of MRI-X-ray-NIR agents are known in the art. For example, the present contrast agents may be formulated as is a sterile aqueous colloid of superparamagnetic particles associated with amphiphilic molecules for intravenous (i.v.) administration. Each milliliter of solution may contain e.g., 10-12 milligrams of coated metal-graphite and 50-70 milligrams of mannitol at a pH of 5 to 9. The formulation may also contain dextran (5.6-9.1 mg/mL) and citrate (0.25-0.53 mg/mL). The osmolarity can be approximately 300-400 mOsm/kg; specific gravity is about 1-1.10.

The dosage may be determined through routine experimentation, and may be expected to be about 0.01 to 100 μM of metal/GC per kilogram of body weight, that is diluted in about 100 mL of 5% dextrose solution and given over about 30 minutes. The diluted drug is preferably administered through a 5-micron filter at a rate of 2 to 4 milliliters per minute.

### XI. TUMOR IMAGING WITH 7 NM FECO/GC

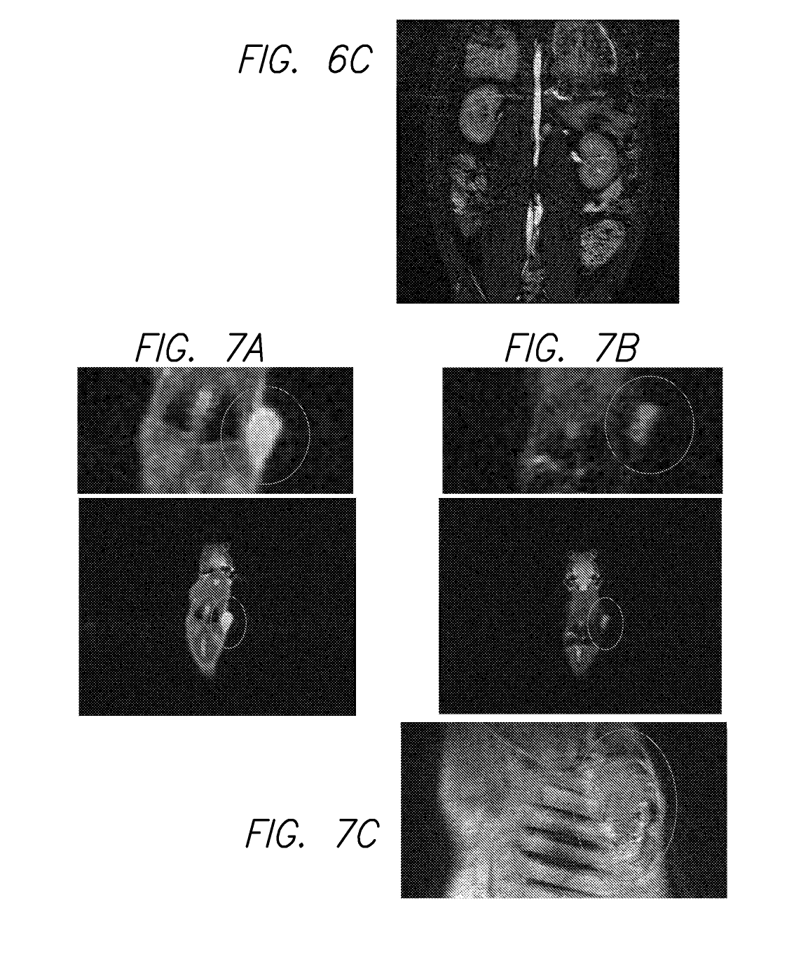

FIG. 7 shows tumor images obtained using the present ˜7 nm FeCo/GC-PL-PEG nanoparticles in a Balb/c mouse implanted with a tumor. FIG. 7A on the left shows a T₂-weighted fast-spin-echo image of a tumor bearing mouse before contrast injection; FIG. 7B on the right shows the image of the tumor approximately 24 hours after the injection. Apparent signal decrease due to contrast uptake can be observed. FIG. 7C shows a high resolution T₁-weighted image showing vascularization around the tumor. The image was obtained with a spectral-spatial excitation for fat suppression with a 3D stack-of-spiral SPGR sequence.

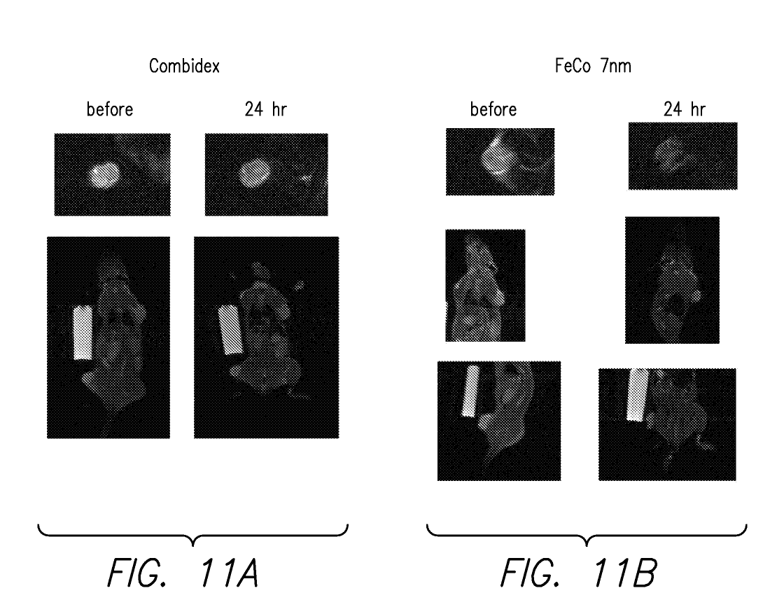

Similarly, FIG. 11 shows T₂-weighted fast spin echo images of a tumor implanted in a mouse. The higher resolution images (top row in 11A and B) were acquired with a 127 ms T_{E} and 4750 ms T_{R} while the images showing the whole mouse were acquired with 40 ms TE and 3000 ms T_{R}. The top row images were acquired with higher resolution (0.015×0.015×0.6 um³ compared to 0.6×0.6×1 mm³ of the bottom images) and more T₂ weighting (longer TE). Combidex® images show no apparent signal change 24 hr after the injection, while the FeCo/GC nanoparticles show significant change in signal intensity. That is, Combidex® has no significant uptake of contrast while the FeCo/GC functionalized material shows uptake (signal darkening).

### XII OTHER NANOCRYSTAL METAL COMBINATIONS

Other metal nanoparticles have been prepared and characterized. These all were prepared with the graphitic coat. Examination by TEM and HRTEM of 10 nm FeCo, 3 nm Co, FeRu. CoRu, FeCoRu, FePt, CoPt and FeCoPt showed regular spheres with HTREM indicating the above-described thin (˜1-2 atom layer) graphitic coats. XRD patterns of these GC nanocrystals showed expected intensity peaks I the 20 (deg) reflection pattern. Magnetization curves as illustrated in FIG. 3A showed comparability between FePt, CoPT and FeCoPt, and similarity to results in FIG. 3A.

### XIII CONJUGATING METAL GRAPHITE NANOCRYSTALS TO DRUGS

The present metal-graphite nanocrystals may be functionalized for in vivo administration, as described above, and further coupled to a biologically active agent, i.e., a drug. The term “drug” is meant to include a variety of agents, such as antibodies (including antibody variants such a minibodies, FAb fragments, etc.) peptide ligands, nucleic acids, and small molecules.

The preparation of the drug-PEG-metal-graphite nanocrystal was substantially as described in Provisional Patent Application Ser No. 60/962,192, filed Jul. 27, 2007, entitled “Supramolecular Functionalization of Nanoparticles for Drug Delivery,” which is specifically incorporated herein by reference in its entirety.

Briefly, that patent application describes in its examples the functionalization of carbon nanotubes. The present graphitic coating, which is extremely robust, tightly bonded directly to the metal, and in graphitic form, can similarly be linked to polymers useful for solubilizing the metal-graphite nanocrystals. PEG is the preferred example.

The hydrophilic polymer (e.g., PEG or dextran) may be covalently bound to the nanoparticle, or adsorbed to it by supramolecular chemistry, such as hydrophobic forces or 7′-stacking.

The small molecule active agents to be delivered by the present materials may be selected from a group of small molecule drugs comprising a fused aromatic ring structure. This permits π-stacking to the aromatic structure of the nanoparticle. In certain embodiments, the active agent is selected from the group consisting of: doxorubicin (DOX), daunorubicin, fluorescein and paclitaxel (PTX). The active agent may be selected from the class of anthracycline-based drugs (e.g., DOX). The active agent may be one of a number of anticancer drugs, which may be generally defined as antiproliferative agents, cytotoxic agents and immunosuppressive agents, e.g., toxorubicin, vinca-alcaloide, actinomycin, toposites, tamoxifen, cisplatin, carboplatin, satraplatin etc.

The present complexes may exhibit a high degree of loading with small molecule active agent.

The metal-graphite nanocrystals used here were essentially functionalized non-covalently by a surfactant of phospholipids-PEG (˜120 polyethylene-oxide PEO units) or covalently by PEGylation (˜220 PEO units) of —COOH groups on graphitic shells generated by refluxing in 2.6M nitric acid. After simple mixing of PEG-FeCo/GC solution with doxorubicin (DOX) at pH=9 overnight and then repeated filtering to remove free DOX in solution, we observed the formation of bound FeCo/GC-DOX complexes. FeCo/GC nanocrystals essentially were loaded with both paclitaxel (PTX) linked to a hydrophilic polymer containing 11 ethylene repeats and having a free amine terminus for conjugation to PTX, and a pyrene terminus for supramolecular bonding to the FeCo/GC nanocrystal. A cleavable linkage is provided between the PTX and the PEG. A variety of cleavable linkages may be employed in order to provide intracellular or circulatory release of the active agent from the PEG or other hydrophilic polymer. An example of a disulfide cleavable linkage is given in J. Am. Chem. Soc. 2005, 36, 12492-12493).

### Making Pyrene-PEG(11)-NH2 for Step Py-PEG Loading

Reagents: 1) 40 mM-NH₂—PEG(11)-BOC, i.e., NH₂—PEG(11)-BOC aka O-(2-Aminoethyl)-O′-[2-(Boc-amino)ethyl]decaethylene glycol—Sigma-Aldrich # 77090>90% purity MW=644.79; 2) 8 mM-1-pyrenebutyric acid MW, Sigma-Aldrich # 257354 MW=288.34; 3) 200 mM-EDC, aka 1-Ethyl-3-[3-dimethylaminopropyl]carbodiimide hydrochloride—pierce #22980 MW=191.7; 4) 50 mM-NHS, N-hydroxysuccinimide-Pierce # 24500 MW=15.09, 5) Methanol, 6) Trifluoroacetic acid (TFA) 100%.

Procedure: Dissolve reagents 1-4 in dry methanol (usually 3 mL scale); mix to react overnight (12-16 hours); dry methanol with house air (rotovap would probably work but never tried); add equal volume of 100% TFA to deprotect BOC group, overnight with mixing (12-16 hrs); dry TFA with house air; add half equivalent volume of methanol (usually 1.5-2 mL) and dry with air, repeat for total of 3×—should give slightly yellow viscous liquid; dissolve product in full equivalent volume of water—should give approx. 8 mM pyrene-PEG(11)-NH₂— transparent, slightly yellow solution, stable for weeks at 4° C.

Functionalizing FeCo/GC with pyrene-PEG(11)-NH2

Procedure: (1) Mix 200-300 nM metal-graphite nanocrystals in water and 1 mM pyrene-PEG(11)-NH2 (typical scale 500-1000 uL). Allow co-functionalization to proceed at 4° C. overnight (12-16 hrs); (2) Filter to remove excess pyrene-PEG(11)-NH₂ through 100 kDa ultracentrifuge filters at 10,000 g. To double check thorough excess removal, one may use a 10 kDa filter and take UV/Vis spectrum of filtrate at 343 nm. Quantify pyrene-PEG(11)-NH₂ loading by UV/Vis Pyrene-PEG(11)-NH₂ ∈≅39,000 M⁻¹cm⁻¹ at 343 nm and Hipco FeCo/Gc∈≅0.0079 nM⁻¹ cm⁻¹ at 808 nm [pyrene-PEG(11)-NH₂]/[FeCO/GC]=# pyrene-PEG(11)-NH₂ per NT=700˜1000

### Paclitaxel Conjugation to Loaded Pyrene-PEG(11)-NH2

Modification of paclitaxel:
50 mg paclitaxel and 90 mg succinic anhydride were mixed in pyridine and reacted for 4 hours. After evaporate the pyridine, water was added and stirred for 20 minutes. Solid was collected at dissolve in a small amount of acetone. Water was added again to precipitate the paclitaxel. Paclitaxel-COOH was freezing dried and stored at −20° C.

Paclitaxel-COOH conjugation on FeCo/GCs.
FeCo/GC solution with PL-PEG-NH2 functionalization loading was reacted with Paclitaxel-COOH (in DMSO) in the presence of EDC and sulfo-NHS. The final concentrations are: 0.5 mM Paclitaxel-COOH, 5 mM EDC, 5 mM sulfo-NHS, 10% DMSO. After overnight reaction at 4 C, excess reagents were removed by filtration through 5 kD filters and washed thoroughly.

In the presently preferred embodiment, the hydrophilic polymer (e.g., PEG) is used conjugated to a hydrophobic polymer for binding to the graphitic shell at one end, and the other end is used for coupling to the drug. That is, for example, amino PEG, which is commercially available, is bonded to the metal graphite nanocrystal (preferably through a phospholipid, as shown in FIG. 3B). This leaves an available amine group at or near the end of the PEG on one or several branches of the PEG. The amine group can be coupled to carboxyl or other groups on the drug of choice.

### XIV. DRUG DELIVERY WITH METAL-GRAPHITE NANOCRYSTALS

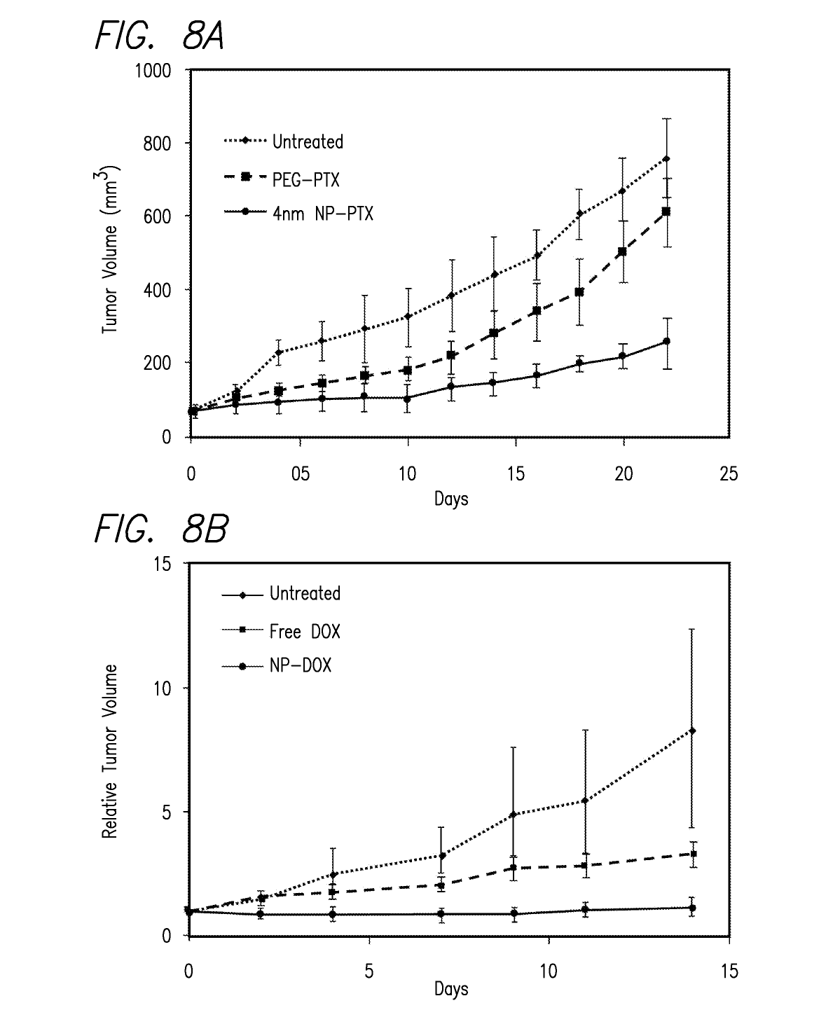

FIG. 8 shows results from two different experiments where FeCo/GC nanocrystals were solubilized with PL-PEG and coupled to a drug. In FIG. 8A, the drug was paclitaxel (PTX). In FIG. 8B, the drug was doxorubicin that is attached to the graphitic shell via π-stacking. Doxorubicin is an anthracycline antibiotic member of the quinine class of anticancer drugs. It contains aromatic rings, which were found to bond to the graphite shell of the present metal graphite nanocrystals. It can be seen from FIG. 8 that tumor volume in an implanted tumor in a mouse model was significantly reduced by the metal-graphite nanocrystal, which was solubilized and bond to an anti-tumor drug.

In FIG. 8A, 4 nm FeCo/graphite nanoparticles were coupled to Paclitaxel (PTX) conjugated to the branched polyethylene glycol (PEG) chains via cleavable ester bond and the branched PEG chains were attached to the graphitic shell through hydrophobic bonding with the lipid portion of a phospholipid. Attachment was accomplished through coupling of PEG to a phospholipid; the lipid portion adsorbed onto the graphite through hydrophobic bonding between the lipid and the graphite. Balb/c mice bearing 4 T₁ murine mammary carcinoma tumor were treated with free PTX or PTX conjugated nanoparticles at a PTX dose of 5 mg/kg every 6 days. 4 T₁ cells may be obtained from the American Type Culture Collection. The tumor sizes were monitored over time. NP-PTX showed significantly better treatment efficacy compared with that of free PTX.

In FIG. 8B, 4 nm FeCo/GC PEG solubilized nanocrystals were used for doxorubicin (DOX) treatment. SCID mice bearing Raji tumor cells (human Burkitt's lymphoma cell line, ATCC CCL-86) were treated with free DOX or DOX loaded by π-stacking on 4 nm FeCo/graphite nanoparticles at a DOX dose of 5 mg/kg every week. In this case, the drug was directly bonded to the graphitic shell through π-stacking between the aromatic rings of the drug and the rings of carbon in the graphite. The tumor sizes were monitored over time. Again NP-DOX showed significantly better treatment efficacy compared with that of free DOX.

### XV. HIGH RESOLUTION IMAGING

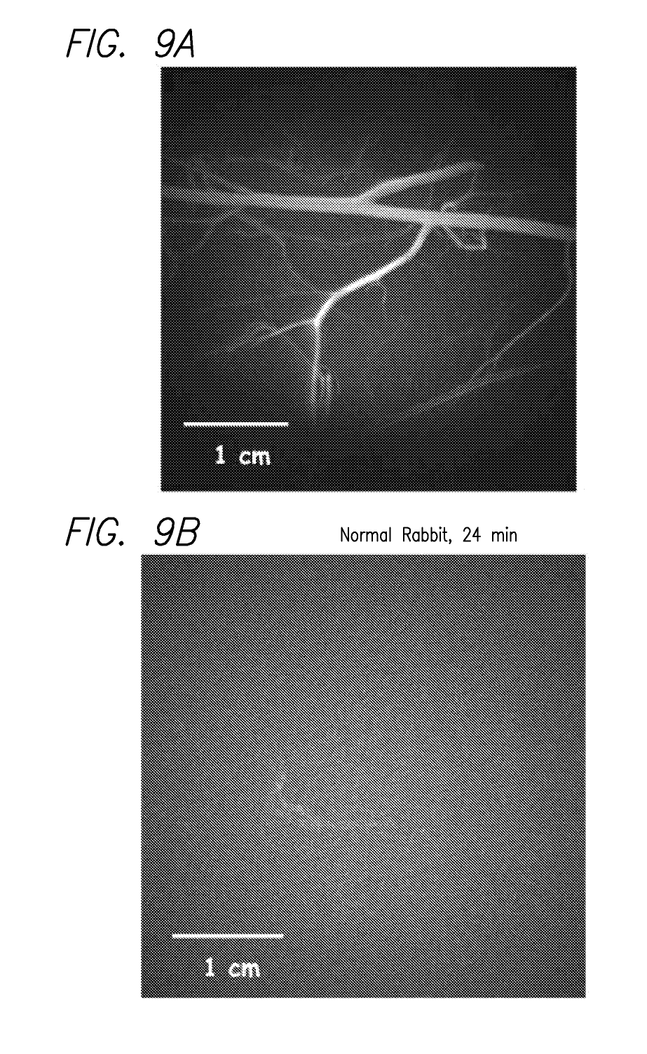

FIG. 9 shows high resolution vascular images of a rabbit hind limb imaged with the present metal graphite nanocrystals and with Combidex® ferumoxtran-10. Combidex® is a synthetic ultrasmall superparamagnetic iron oxide composed of dextran-coated iron oxide nanoparticles (also known as ‘ultrasmall particulate iron oxides’ or USPIO), from Advanced Magnetics, Inc. The high contrast obtained using FeCo/GC nanocrystals give an image with small vasculature while Combidex® fails to give such contrast even at a 5 times higher dosage. The FeCo/GC nanocrystal's capability to deliver micro-vasculature images shows its potential to serve as an important tool for diagnosing vascular diseases in smaller vessels and/or imaging the small vasculature around tumor to monitor treatments. A small surface coil was placed on the hind limb and a 3D stack-of-spirals SPGR image was obtained with a spatial resolution of 78 um×78 um×500 um. FIG. 9A is an image obtained with a ˜9.6 uM/kg injection of 7 nm FeCo/GC nanoparticles while FIG. 9B is obtained with a ˜48 uM/kg injection of Combidex. Note how the high contrast obtained using FeCo/GC nanocrystals give an image with small vasculature while Combidex fails to give such contrast even at a 5 times higher dosage. The FeCo/GC nanocrystal's capability to deliver micro-vasculature images shows its potential to serve as an important tool for diagnosing vascular diseases in smaller vessels and/or imaging the small vasculature around tumor to monitor treatment or classify tumors.

### XVI. SIGNAL ENHANCEMENT VS. CONCENTRATION: SPGR

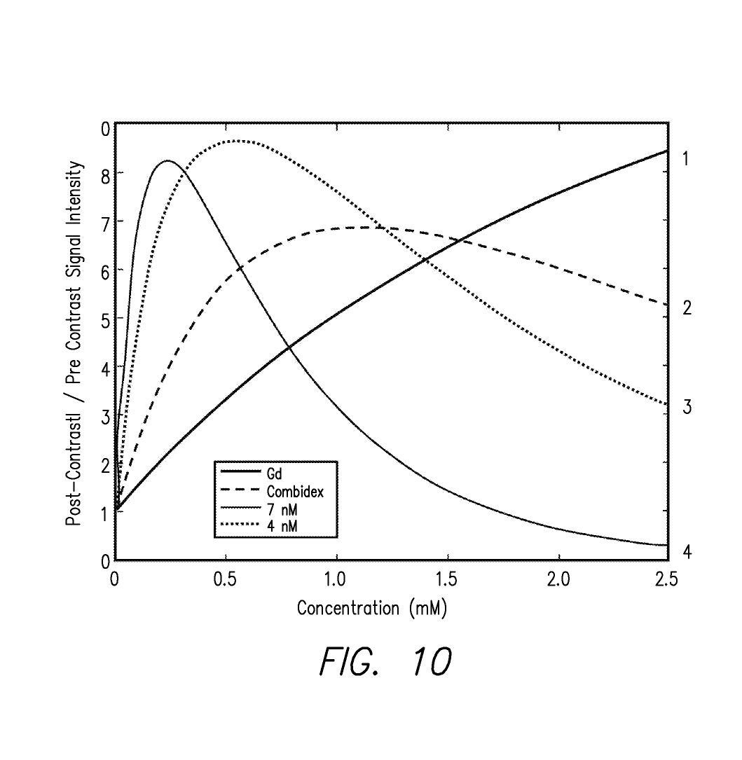

The graph in FIG. 10 shows theoretical calculations of a 3D SPGR signal enhancement for blood. Assuming an initial T₁ and T₂ values of 1200 ms and 327 ms, the ratio of signal enhancement compared to the initial signal intensity is plotted. T_{E} and T_{R} are assumed to be 4 ms and 40 ms. An important thing to note is that for short circulating agents such as Magnevist®, the contrast will start leaking out of the blood vessels in only a few seconds. Therefore, the theoretically predicted signal intensity is only observable for a short amount of time. However, for longer circulating nanocrystal based agents, the enhancement can be captured with a reasonable scan time. The 7 nm FeCo/GC shows significant enhancement even at a very low concentration (˜0.2 mM).

Further details on obtaining SPGR imaging may be found in US Patent Application Publication 2005/0256393, by Deoni, et al., published Nov. 17, 2005, entitled “System and method for generating t₁ and t₂ maps.”

### XVII. ALTERNATIVE EMBODIMENTS AND VARIATIONS

There are many possible variations and modifications of the methods described experimentally above. The sizes of nanocrystals can be modified. This is important for achieving specific relaxation properties in conjunction with specific biodistribution since particle size changes both the relaxation properties and the biological distribution. Sizes can vary between 2 nm to tens of nanometers.

The stability and distribution of the particles in-vivo can also be modified by changing the functional materials, specifically the length and branching of PEG and the functional groups of functional materials. Modifying the functional material can be aimed towards increasing circulation time without being taken up by a specific organ or targeting specific disease areas such as cancer or atherosclerosis. Various covalent and noncovalent functionalization schemes can be developed for the graphitic shell on the FeCo core.

It is also possible to incorporate (or replace) one or more additional elements into the metal core of the nanoparticles to improve the X-ray absorption efficiency and/or to enhance other imaging modalities. For example, by adding high magnetic susceptibility metal(s) such as Gadolinium and Terbium in the nanoparticle, it is likely that the agent will enhance MR T₁ images, and by adding other metal(s) or replacing Au with other metal(s), similar or better enhancement in X-ray (or alike) imaging is possible.

The use of metals like gold and/or iron as alloys in the present nanoparticles is also specifically contemplated. While the use of iron-oxide nanoparticles for enhancing MRI contrast, is known, little has been reported in the literature on the use of heavy element nanoparticles as x-ray contrast agents, nor the use of composite nanoparticles for dual or multi-modality imaging. The present development of metal-carbon core-shell nanoparticles according to the present invention involves the idea of using heavy element nanoparticles as x-ray contrast agent, the choice of Au as one element of the nanoparticles, the use of composite nanoparticles for dual x-ray and MR image enhancement, the method of synthesis, and the determination of the optimal parameters. Because of the higher atomic number of Au compared to iodine or barium, which forms the basis for current x-ray contrast agents, the new agents disclosed herein which contain Au are potentially more efficient than these agents. Au is a preferred material because it has high electron density, efficient Compton scattering; and a high Z, probability of photoelectric absorption per unit mass is approximately proportional to Z3/E3. Also, Au is an efficient absorption medium for diagnostic and therapeutic X-rays, and has low toxicity. Also, FeRu nanocrystals having the present graphitic coating have been made, at a diameter of 2 nm.

Combining Au and Fe will produce a dual modality imaging agent (MRI and CT) and therapeutic agent useful in magnetic hyperthermia therapy and radiation therapy. The graphitic carbon shell will prevent phase separation of Au and Fe at room temperature. Another advantage is the low toxicity of carbon.

Further applications include use of the present nanoparticles as CT contrast agents having higher retention rate and tissue-specificity. Because of the in-vivo heating effect of magnetic nanoparticles, the present agents may be used for hyperthermic therapy as well. Since these agents are good X-ray absorption medium, it can be used for radiation therapy as well. Therefore, it is possible that this agent functions as a one-does-all agent. The present nanocrystals may be selected from materials having a high X-Ray Mass Attenuation Coefficient for the energy of the radiation being used. Such information may be obtained from NIST, e.g., at http://physics.nist.gov/PhysRefData/XrayMassCoef/tab3.html.

The present hydrophilic functionalization scheme, using phospholipid-PEG may be modified as described. Hydrophilic polymers may be covalently attached to the graphite shell using diazonium chemistry. This reaction may be carried out as described, e.g., in Pan et al., “Natural graphite modified with nitrophenyl multilayers as anode materials for lithium ion batteries,” J. Mater. Chem., 2007, 17, 329-334, and references cited therein.

### CONCLUSION

The present examples, methods, procedures, specific compounds and molecules are meant to exemplify and illustrate the invention and should in no way be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but would be understood by workers in the field. Such patents or publications are hereby incorporated by reference to the same extent as if each was specifically and individually incorporated by reference and for the purpose of describing and enabling the method or material referred to.

### REFERENCES

1. Bardos, D. I. Mean magnetic moments in bcc Fe—Co alloys. J. Appl. Phys. 40, 1371-1372 (1969).
2. Hütten, A. et al., New magnetic nanoparticles for biotechnology. J. Biotechnol. 112, 47-63 (2004).
3. Reiss, G. & Hütten, A Magnetic nanoparticles: Applications beyond data storage. Nat. Mater. 4, 725-726 (2005).
4. Lewin, M. et al., Tat peptide-derivatized magnetic nanoparticles allow in vivo tracking and recovery of progenitor cells. Nature Biotech. 18, 410-414 (2000).
5. Alivisatos, P. The use of nanocrystals in biological detection. Nat. Biotechnol. 22, 47-52 (2004).
6. Kim, S. et al., Near-infrared fluorescent type II quantum dots for sentinel lymph node mapping. Nature Biotech. 22, 93-97 (2004).
7. Hirsch, L. R. et al., Nanoshell-mediated near-infrared thermal therapy of tumors under magnetic resonance guidance. Proc. Natl. Acad. Sci. USA 100, 13549-13554 (2003).
8. Liao, H. W. & Hafner, J. H. Gold nanorod bioconjugates. Chem. Mater. 17, 4636-4641 (2005).
9. Huang, X., El-Sayed, I. H., Qian, W. & El-Sayed, M. A. Cancer cell imaging and photothermal therapy in the near-infrared region by using gold nanorods. J. Am. Chem. Soc. 128, 2115-2120 (2006).
10. Chen, C.-C. et al., DNA-gold nanorod conjugates for remote control of localized gene expression by near infrared irradiation. J. Am. Chem. Soc. 128, 3709-3715 (2006).
11. Kopelman, R. et al., Multifunctional nanoparticle platforms for in vivo MRI enhancement and photodynamic therapy of a rat brain cancer. J. Magnetism and Mag. Mater. 293, 404-410 (2005).
12. McCarthy, J. R., Jaffer, F. A. & Weissleder, R. A Macrophage-Targeted Theranostic Nanoparticle for Biomedical Applications. Small 2, 983-987 (2006).
13. Dinega, D. P. & Bawendi, M. G. A solution-phase chemical approach to a new crystal structure of cobalt. Angew. Chem. Int. Ed. 38, 1788-1791 (1999).
14. Puntes, V. F., Zanchet, D., Erdonmez, C. K. & Alivisatos, A. P. Synthesis of hcp-Co nanodisks. J. Am. Chem. Soc. 124, 12874-12880 (2002).
15. Sun, S., Murray, C. B., Weller, D., Folks, L. & Moser, A. Monodisperse FePt nanoparticles and ferromagnetic FePt nanocrystal superlattices. Science 287, 1989-1992 (2000).
16. Park, J. et al., Ultra-large-scale syntheses of monodisperse nanocrystals. Nat. Mater. 3, 891-895 (2004).
17. Desvaux, C. et al., Multimillimetre-large superlattices of air-stable iron-cobalt nanoparticles. Nat. Mater. 4, 750-753 (2005).
18. Turgut, Z., Scott, J. H., Huang, M. Q., Majetich, S. A. & McHenry, M. E. Magnetic properties and ordering in C-coated Fe{sub x}Co{sub 1-x} alloy nanocrystals. J. Appl. Phys. 83, 6468-6470 (1998).
19. Bai, J. & Wang, J.-P. High-magnetic-moment core-shell-type FeCo—Au/Ag nanoparticles. Appl. Phys. Lett. 87, 152502 (2005).
20. Tuinstra, F. & Koenig, J. L. Raman spectrum of graphite. J. Chem. Phys. 53, 1126-1130 (1970).
21. Lu, Y., Zhu, Z. & Liu, Z. Carbon-encapsulated Fe nanoparticles from detonation-induced pyrolysis of ferrocene. Carbon 43, 369-374 (2005).
22. Li, Y. et al., Growth of Single-Walled Carbon Nanotubes from Discrete Catalytic Nanoparticles of Various Sizes. J. Phys. Chem. 105, 11424-11431 (2001).
23. Kam, N. W. S., Liu, Z. & Dai, H. Functionalization of carbon nanotubes via cleavable disulfide bonds for efficient intracellular delivery of siRNA and potent gene silencing. J. Am. Chem. Soc. 127, 12492-12493 (2005).
24. Kam, N. W. S., O'Connell, M., Wisdom, J. A. & Dai, H. Carbon nanotubes as multifunctional biological transporters and near-infrared agents for selective cancer cell destruction. Proc. Natl. Acad. Sci. USA 102, 11600-11605 (2005).
25. Cataldo, F. An investigation on the optical properties of carbon black, fullerite, and other carbonaceous materials in relation to the spectrum of interstellar extinction of light. Full, Nanotubes, & Carb. Nanostructures 10, 155-170 (2002).
26. Jung, C. W. & Jacobs, P. Physical and chemical properties of superparamagnetic iron oxide MR contrast agents: ferumoxides, ferumoxtran, ferumoxsil. Magn. Reson. Imag. 13, 661-674 (1995).
27. Kellar, K. E. et al., NC100150 injection, a preparation of optimized iron oxide nanoparticles for positive-contrast MR angiography. J. Mag. Res. Imaging 11, 488-494 (2000).
28. Huang, D.-M. et al., Highly efficient cellular labeling of mesoporous nanoparticles in human mesenchymal stem cells: implication for stem cell tracking. FASEB J. 19, 2014-2016 (2005).
29. Kam, N. W. S. & Dai, H. Carbon nanotubes as intracellular protein transporters: Generality and biological functionality. J. Am. Chem. Soc. 127, 6021-6026 (2005).
30. Bianco, A., Kostarelos, K., Partidos, C. D. & Prato, M. Biomedical applications of functionalised carbon nanotubes. Chem. Commun., 571-577 (2005).

What is claimed is:

1. A nanocrystal comprising metal in at least one graphitic shell.

2. The nanocrystal of claim 1 wherein said metal is substantially free of oxygen.

3. The nanocrystal of claim 1 wherein the metal is selected from the group consisting of Fe, Co, Ni, Au, Pt, FeCo, FeNi, FeAu, FePt, FeRu, CoRu, FeCoRu, FeCoPt, and CoPt.

4. The nanocrystal of claim 1 wherein the metal is of the formula XY and optionally Z, where X, Y and Z are the same or different each independently selected from the group consisting of: Fe, Co, Ni, Au, Pt, Co, Ni, Ru, and Pt.

5. The nanocrystal of claim 1 wherein said graphitic shell comprises between 1 and 10 graphitic layers.

6. The nanocrystal of claim 5 having no more than 2 graphitic layers.

7. The nanocrystal of claim 1 which is about 2 to 7 nm in diameter.

8. The nanocrystal of claim 1 which is less than about 25 nm in diameter.

9. The nanocrystal of claim 1 which is superparamagnetic or ferromagnetic.

10. The nanocrystal of claim 9 further comprising a coating of organic molecules.

11. The nanocrystal of claim 10 wherein said organic molecules are organic amphiphilic molecules noncovalently bound to the graphitic shell

12. The nanocrystal of claim 10 wherein the organic amphiphilic molecules further comprise a polar lipid adsorbed onto the graphitic shell.

13. The nanocrystal of claim 12 wherein the polar lipid is linked to a hydrophilic polymer selected from the group consisting of PEG, dextran or polyglycerol.

14. The nanocrystal of claim 13 where the hydrophilic polymer is PEG.

15. The nanocrystal of claim 14 wherein the PEG is branched and has a molecular weight between 200 and 20,000.

16. The nanocrystal of claim 10 wherein said organic molecules are covalently bonded to the graphitic shell.

17. The nanocrystal of claim 15 wherein the metal is selected from the group consisting of AuFe, FeCo and FeRu.

18. The nanocrystal of claim 1 wherein the metal is a single crystal.

19. The nanocrystal of claim 1 wherein the magnetic saturation is at least 160 emu/g.

20. A method of labeling cells comprising contacting the cells with a nanocrystal comprising a metal in at least one graphitic shell, whereby the nanocrystal is absorbed by the cell and responds to an electromagnetic field.

21. A method of labeling cells comprising contacting the cells with a superparamagnetic nanocrystal which comprises metal in at least one graphitic shell, wherein said shell is attached to an organic molecule, whereby the nanocrystal is absorbed by the cell and responds to an electromagnetic field.

22. The method of claim 21 wherein the organic molecule is an amphiphilic molecule.

23. The method of claim 22 wherein the amphiphilic molecule comprises a polar lipid linked to PEG.

24. The method of claim 21 wherein the nanocrystal comprises Fe and further contains a phospholipid-PEG coating.

25. A method of heating cells comprising contacting the cells with a nanocrystal comprising metal in at least one graphitic shell, comprising the step of applying radiation to the cells, whereby the radiation is absorbed by the graphitic shell and heats the cells containing them.

26. The method of claim 25 wherein the radiation is selected from NIR and magnetic.

27. The method of claim 25 wherein said metal comprises between one and three elements of a Group VIII transition metal.

28. The method of claim 25 wherein the metal is selected from the group consisting of Fe, Co, Ni, Au, Pt, FeCo, FeNi, FeAu, and FePt.

29. An MRI contrast agent formulation, suitable for injection into a mammalian host, comprising a superparamagnetic nanocrystal comprising metal in a graphitic shell with a coating of a hydrophilic organic molecule.

30. The contrast agent formulation of claim 29 wherein the metal comprises iron.

31. The contrast agent formulation of claim 29 wherein the metal comprises either gold or cobalt.

32. The contrast agent formulation of claim 29 wherein the metal is selected from the group consisting of FeAu and FeCo.

33. The contrast agent formulation of claim 29 having a Fe to Co ratio between 10:90 and 90:10.

34. A method of imaging a subject comprising the steps of administering to the subject the contrast agent formulation of claim 29, applying a magnetic field to the subject, and obtaining images showing magnetic field alterations caused by the contrast agent.

35. The method of claim 34 wherein the magnetic field alteration comprises an r₁, r₂, or both r₁ and r₂ of the contrast agent, at 1.5 Tesla, of at least 150 mM-1s.-1

36. A dispersed nanocrystal preparation comprising metal in at least one graphitic shell for delivery of an active agent into a cell, comprising:
(a) a hydrophilic polymer bound to the graphitic shell; and
(c) an active agent, attached to the nanocrystal.

37. A nanocrystal according to claim 36 in which the hydrophilic polymer is linked to the active agent.

38. A method of imaging blood flow in a blood vessel by applying a magnetic field to a dispersed preparation of metal-graphite nanocrystals which have been linked to a hydrophilic polymer and injected into the blood vessel.

39. A method of reducing tumor size in a subject having a tumor, comprising the step of administering to the subject a preparation containing a functionalized metal-graphite nanocrystal linked to an anti-tumor drug.
